(19) United States
(12) Patent Application Publication
(10) Pub. No.: US 2004/0023213 A1
(43) Pub. Date: Feb. 5, 2004

(54) Domain specific gene evolution

(75) Inventors: David Zarling, Menlo Park, CA (US); Gurucharan Reddy, Fremont, CA (US); Sushma Pati, Woodside, CA (US)

Correspondence Address: RICHARD F. TRECARTIN, ESQ., FLEHR HOHBACH TEST ALBRITTON & HERBERT LLP, Suite 3400, Four Embarcadero Center, San Francisco, CA, 94111-4187, US

(73) Assignee: Pangene Corporation

(21) Appl. No.: 09/886,171

(22) Filed: Jun. 20, 2001

Related U.S. Application Data
(63) Continuation of application No. 09/373,347, filed on Aug. 12, 1999, now abandoned.
Application No. 09/373,347 is a continuation-in-part of application No. 09/133,934, filed on Aug. 14, 1998, now Pat. No. 6,074,853.
(60) Provisional application No. 60/096,330, filed on Aug. 12, 1998.

(51) Int. Cl. C12Q 1/68; C07H 21/04; C12P 19/34

(52) U.S. Cl. 435/6; 435/91.2; 435/440; 536/23.1

(57) ABSTRACT

Compositions and methods are provided for rapidly evolving specific protein domains employing a recombinase and recombination intermediates.

This is a continuing application of U.S. application Ser. No. 60/096,330 filed Aug. 12, 1998, pending and U.S. application Ser. No. 09,133,934 filed Aug. 14, 1998, pending.

### FIELD OF THE INVENTION

The invention relates to compositions and methods of rapidly evolving specific protein domains using a library of nucleic acid filaments and a recombinase polypeptide or peptide.

### BACKGROUND OF THE INVENTION

In nature, the evolution of genes and their encoded proteins occurs through an equilibrium between recombination or mutation and selection. While evolution in nature takes millions of years, in vitro methods and compositions have been developed to evolve proteins, with improved and novel functions, in a matter of hours to days.

Current in vitro gene evolution methods utilize repeated cycles of random mutagenesis or random nicking and mixing of related genes containing mutations in PCR-based random recombination. These methods couple multiple rounds of in vitro mutagenesis with screening systems to produce and identify the desired mutants or recombinants (Stemmer 1994. Nature 370:389-391; Arnold 1996. Chemical Engineering Science 51:5091-5102). Research has shown, however, that the mutations of interest tend to occur in those regions or domains that are directly related to function (Chen and Arnold. 1993. PNAS USA 90:5618-5622). However, these mutagenesis methods produce random mutations throughout the gene of interest which requires the need to screen large numbers of uninteresting or deleterious mutants. The labor-intensive and time consuming aspects of these methods are further complicated by the necessity of multiple rounds of subcloning and can be extremely challenging if the screening system is complex and does not utilize a selection system.

Homologous recombination (HR) is defined as the exchange of homologous or similar DNA sequences between two DNA molecules. As essential feature of HR is that the enzymes responsible for the recombination event can pair any homologous sequences as substrates. The ability of HR to transfer genetic information between DNA molecules makes targeted homologous recombination a very powerful method in genetic engineering and gene manipulation. HR can be used to add subtle mutaitons at known sites, replace wild type genes or gene segments or introduce completely foreign genes into cells. However, HR efficiency is very low in living cells and is dependenet on several parameters, including the method of DNA delivery, how it is packaged, its size and conformation, DNA length and position of sequences homologous to the target, and the efficiency of hybridization and recombination at chromosomal sites. These variables severely limit the use of conventional HR approaches for gene evolution in cell based systems. (Kucherlapati et al., 1984. PNAS USA 81:3153-3157; Smithies et al. 1985. Nature 317:230-234; Song et al. 1987. PNAS USA 84:6820-6824; Doetschman et al. 1987. Nature 330:576-578; Kim and Smithies. 1988. Nuc. Acids. Res. 16:8887-8903; Koller and Smithies. 1989. PNAS USA 86:8932-8935; Shesely et al. 1991. PNAS USA 88:4294-4298; Kim et al. 1991. Gene 103:227-233).

The frequency of HR is significantly enhanced by the presence of recombinase activities in cellular and cell free systems. Several proteins or purified extracts that promote HR (i.e., recombinase activity) have been identified in prokaryotes and eukaryotes (Cox and Lehman, 1987. Annu. Rev. Biochem. 56:229-262; Radding. 1982. Annual Review of Genetics 16:405-547; McCarthy et al. 1988. PNAS USA 85:5854-5858). These recombinases promote one or more steps in the formation of homologously-paired intermediates, strand-exchange, and/or other steps. The most studied recombinase to date is the RecA recombinase of E. coli, which is involved in homology search and strand exchange reactions (Cox and Lehman, 1987, supra).

The bacterial RecA protein (Mr 37,842) catalyses homologous pairing and strand exchange between two homologous DNA molecules (Kowalczykowski et al. 1994. Microbiol. Rev. 58:401465; West. 1992. Annu. Rev. Biochem. 61:603-640); Roca and Cox. 1990. CRC Cit. Rev. Biochem. Mol. Biol. 25:415-455; Radding. 1989. Biochim. Biophys. Acta. 1008:131-145; Smith. 1989. Cell 58:807-809). RecA protein binds cooperatively to any given sequence of single-stranded DNA with a stoichiometry of one RecA protein monomer for every three to four nucleotides in DNA (Cox and Lehman, 1987, suspra). This forms unique right handed helical nucleoprotein filaments in which the DNA is extended by 1.5 times its usual length (Yu and Egelman 1992. J. Mol. Biol. 227:334-346). These nucleoprotein filaments, which are referred to as DNA probes, are crucial “homology search engines” which catalyze DNA pairing. Once the filament finds its homologous target gene sequence, the DNA probe strand invades the target and forms a hybrid DNA structure, referred to as a joint molecule or D-loop (DNA displacement loop) (McEntee et al. 1979. PNAS USA 76:2615-2619; Shibata et al. 1979. PNAS USA 76:1638-1642). The phosphate backbone of DNA inside the RecA nucleoprotein filaments is protected against digestion by phosphodiesterases and nucleases.

RecA protein is the prototype of a universal class of recombinase enzymes which promote probe-target pairing reactions. Recently, genes homologous to E. coli RecA (the Rad51 family of proteins) were isolated from all groups of eukaryotes, including yeast and humans. Rad51 protein promotes homologous pairing and strand invasion and exchange between homologous DNA molecules in a similar manner to RecA protein (Sung. 1994. Science 265:1241-1243; Sung and Robberson. 1995. Cell 82:453461; Gupta et al. 1997. PNAS USA 94:463-468; Baumann et al. 1996. Cell 87:757-766).

Methods and compositions that have been used to target and alter, by homologous recombination, substitutions, insertions and deletions in target sequences have been described; see U.S. application Ser. Nos. 08/381,634; 08/882,756; 09/301,153; 08/781,329; 09/288,586; 09/209,676; 09/007,020; 09/179,916; 09/182,102; 09/182,097; 09/181,027; 09/260,624; and internation application nos. U.S. Ser. No. 97/19,324; U.S. Ser. No. 98/26,498; U.S. Ser. No. 98/01,825.

Accordingly, it is an object of the invention to provide an efficient method of domain specific gene evolution that generates maximal diversity but increases the probability of identifying a gene of interest.

### SUMMARY OF THE INVENTION

The present invention provides methods of domain specific gene evolution comprising forming a plurality of recombination intermediates comprising a target nucleic acid encoding an amino acid sequence of interest, a recombinase and a plurality of targeting polynucleotides. The targeting polynucleotides are substantially complementary to each other and each comprises a homology clamp that substantially correspond to or is substantially complementary to a predetermined sequence of the target nucleic acid and comprise random or degenerate sequences. The predetermined sequence encodes a domain of the amino acid sequence. The method further comprises contacting the intermediate with a recombination proficient cell, whereby a library of altered target nucleic acids are produced. The altered target nucleic acids are expressed in the cell to generate a pool of variant amino acid sequences. The method further comprises selecting and isolating a cell comprising an altered target nucleic acid that expressed a variant amino acid having a desired activity.

In another aspect of the invention, a method of domain specific gene evolution comprises forming a recombination intermediate comprising a target nucleic acid encoding an amino acid sequence of interest, a recombinase and a pair of targeting polynucleotides. The targeting polynucleotides are substantially complementary to each other and each comprises a homology clamp that substantially corresponds to or is substantially complementary to a predetermined sequence of the target nucleic acid. The predetermined sequence encodes a domain of the amino acid sequence. The method further comprises contacting the intermediate with a single-strand specific nuclease or junction-specific nuclease to form a nicked or open-ended target nucleic acid. The regions adjacent to the hybridized region or junctions are susceptible to nucleases. The target nucleic acid is reassembled and recombined to produce a library of altered target nucleic acids. The target nucleic acids are expressed to generate a pool of variant amino acid sequences. The variant amino acid sequences are selected and characterized to identify an altered target nucleic acid encoding a variant amino acid sequence of interest.

In a further aspect, each method is repeated one or more times to further evolve a variant amino acid sequence having a desired activity.

In yet another aspect, more than one domain or a protein is evolved simultaneously.

### DETAILED DESCRIPTION OF THE FIGURES

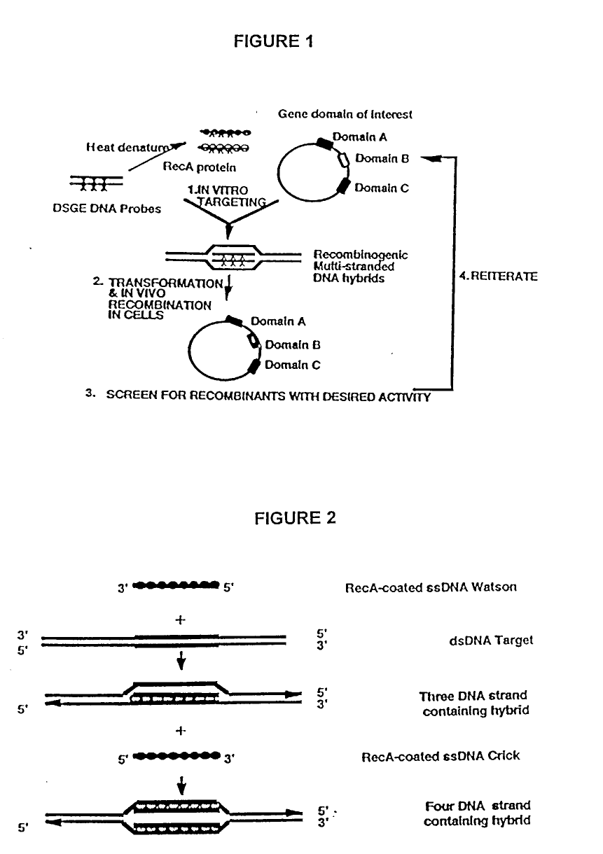

FIG. 1 depicts domain specific gene evolution (DSGE) by recombinase mediated complementary single-strand DNA targeting (cssDNA or targeting polynucleotide). 1) targeting of domain B of the gene of interest with RecA coated cssDNA degenerate probes; 2) deproteinizing and transforming of multistranded recombinogenic DNA hybrid products in bacteria; 3) screening and selection of mutants of itnerest; 4) reiterating of steps 1-3.

FIG. 2 depicts native gene targeeting assay (double D-loop formation): cssDNA strands are generated by heat denaturation of double-stranded DNA PCR products and then coated with RecA protein (shown as circles) in appropriate buffer conditions to form nucleoprotein filaments. To target a homologous site in its native DNA, RecA nucleoprotein filaments can be added to the duplex in a sequential manner or simultaneously. Interaction of one filament with its duplex results in the formation of a three-stranded DNA intermediate (a single D-loop), which is unstable after deproteinization. Addition of a complementary strand stabilizes the otherwise unstable intermediate by forming a kinetically trapped double D-loop. This double D-loop is highly stable after deproteinization, can be manipulated in a numer of different ways and is biologically active.

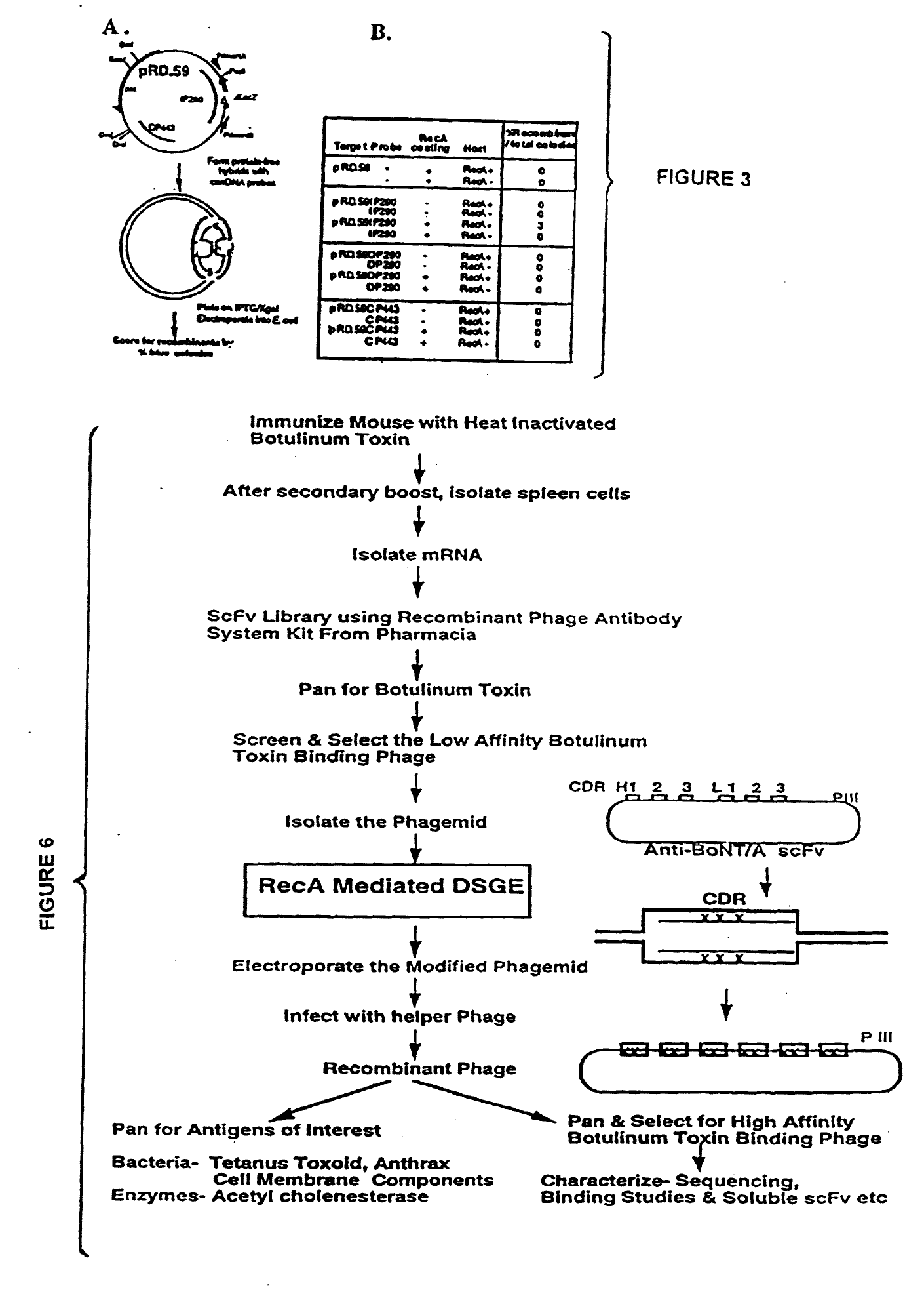

FIGS. 3A-B depicts Probe-target Hybrids Enhance Homologous Recombination in Bacteria. Panel A: the scheme for the EHR assay used in Panel B. Panel B: shows data for the enhanced homologous recombination (EHR) of cssDNA probe:target hybrids in E. coli. The homologously targeted probe:target hybrids have enhanced homologous recombination frequencies in recombination proficient cells. cssDNA probe:target hybrids formed were introduced into RecA+ and RecA− E. coli. The molar ratio of the cssDNA probe:target in the in vitro targeting reaction varied from 1:1 to 1:5.6. The % recombinant/total colonies is the percentage blue colonies in the total population of ampicillin-resistant colonies. Groups with 0% recombinants did not produce any blue colonies in at least 105 plated colonies. Plasmid DNA was isolated from blue colonies that were serially propagated for three generations to determine if homologous recombination stably occurred in the lacZ gene.

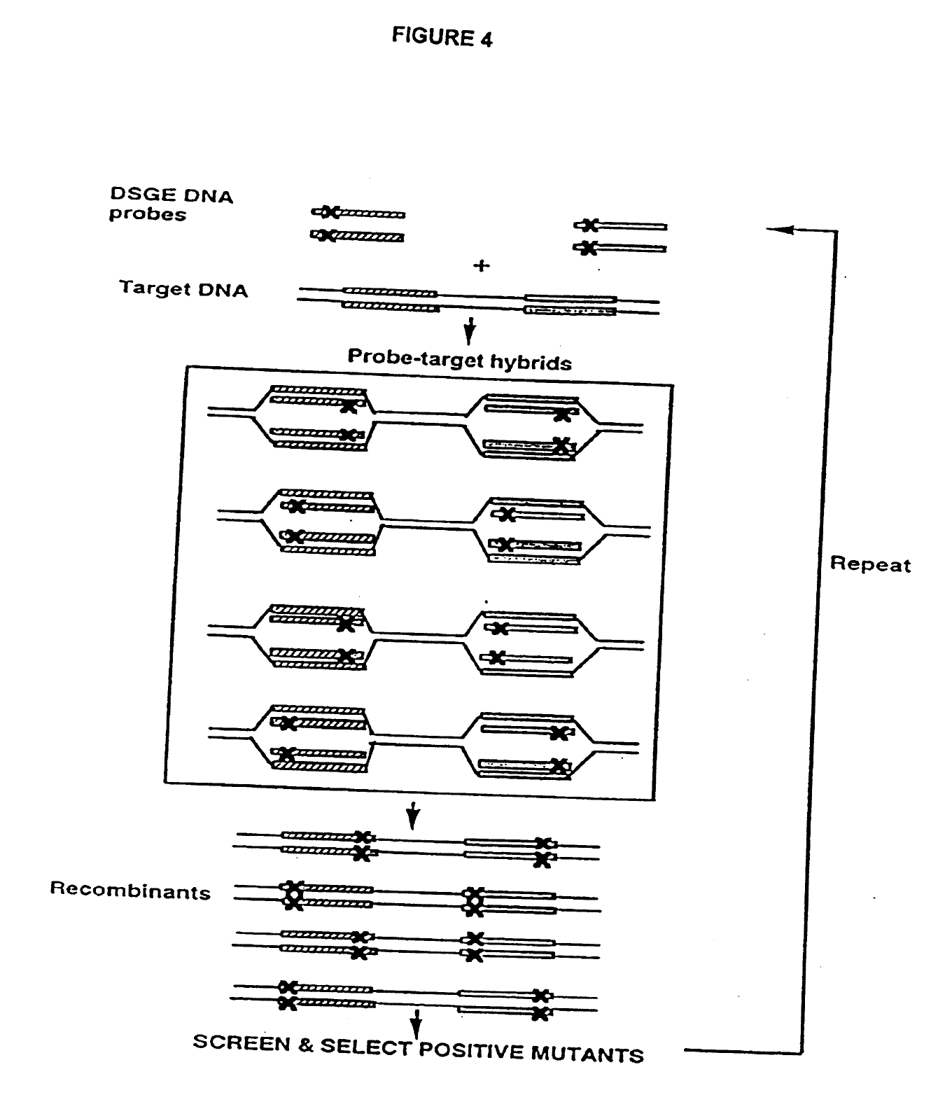

FIG. 4 depicts a design of DSGE DNA Probes. DSGE DNA probes can target and mutagenize either a single domain or multiple domains. These combinatorial DSGE DNA probes contain mutations throughout their length or can be degenerate. The resulting recombinants or mutants, contain many possible combinations of mutations in the targeted domains. In this case targeting and mutagenesis of two domains is shown and RecA protein is not shown for clarity. Single domain shuffling probes target and mutagenize a single region. These probes contain two external homology clamps in which the sequence of the probes corresponds exactly to the sequence in the target. Internal regions contain various degrees of mismatches to mutagenize and shuffle that specific region.

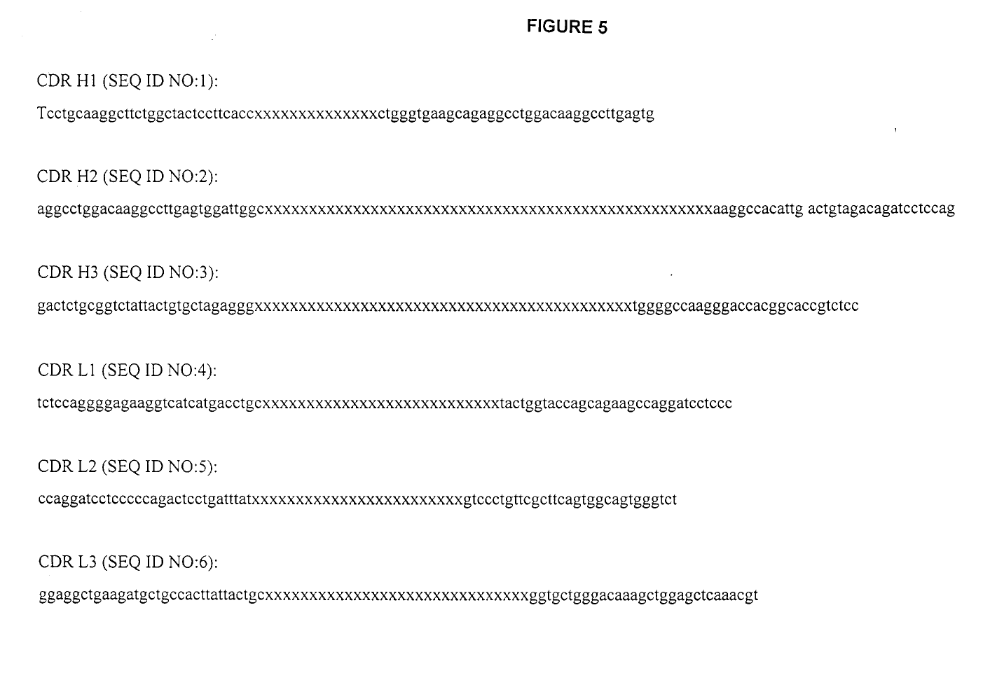

FIG. 5 depicts targeting polynucletides for evolving CDR region of scFv to Botulinum neurotoxin.

FIG. 6 depicts a flow chart of steps in single-chain scFv antibody pahge library evolution by DSGE. Variable regions of both the light and heavy chain are present in one molecule in single-chain Fv. CDR regions are targeted hybrids are transformed into E. coli along with helper phage to allow packaing and expression. After screening to eliminate the non-evolved antibody-phage, the process is reitereated to evolve the antibody-phage library.

### DESCRIPTION OF PREFERRED EMBODIMENTS

The present invention provides methods and compositions for domain specific gene evolution. In one aspect of the invention, the method comprises targeting a predetermined nucleic acid sequence that encodes a specific protein domain, to make a plurality of targeted sequence modifications. That is, by targeting the recombinogenic probes of the invention to particular protein domains, gene evolution and selection are targeted to specific domains known or believed to harbor specific activities or functions. These methods create maximal diversity in specific domains of interest, thereby, decreasing the size of the library of mutations that are to be screened and increasing the probability of finding a gene with improved or desired attributes. Therefore, the libraries of the present invention are enriched for advantageous or interesting mutations or recombinant sequence(s).

Accordingly, the methods comprise combining a plurality of pairs of single-stranded targeting polynucleotides, a predetermined target nucleic acid, and a recombinase to form a polynucleotide:target nucleic acid complex. The targeting polynucleotides comprise at least one homology clamp for targeting a predetermined domain of a target nucleic acid and randomized or degenerate sequences. The complex is optionally introduced into a plurality of recombination proficient cells which catalyze strand exchange and homologous recombination intracellularly to produce a library of modified nucleic acids. Cells are selected and isolated that comprise a modified nucleic acid that encodes a polypeptide having a desired property. The process is preferably repeated iteratively to further evolve the target domain of interest. This process is depicted schematically in FIG. 1.

In another aspect of the invention, methods of domain specific DNA nicking are provided for domain specific gene evolution. This method comprises combining a pair of single-stranded targeting polynucleotides, a predetermined target nucleic acid, and a recombinase to form a polynucleotide:target nucleic acid complex. The targeting polynucleotides are substantially complementary and comprise at least one homology clamp for targeting a predetermined domain of a target nucleic acid. The polynucleotide:target nucleic acid complex is treated with a single-strand specific nuclease, which preferentially nicks the regions flanking the polynucleotide:target nucleic acid complex region (Ferrin and Camerini-Otero. 1991. Science. 254 1494-1497). That is, the domain is protected from recombination by the initial presence of the recombinase in the complex. The nuclease is inactivated and the complex dissociated. The nicked target nucleic acid is reassembled and recombined by PCR to produce a library of nucleic acids with preferential modifications in the nicked regions. The library of modified nucleic acids can be introduced into a host cell and expressed. Cells are selected and isolated that comprise a modified nucleic acid that encodes a polypeptide having a desired property. This process is repeated iteratively to further evolve the predetermined targeted domain of interest.

In each of the methods described above, single domains and optionally multiple domains are targeted.

The methods and compositions described above are optionally used in combination for domain specific gene evolution. For example, individual or multiple rounds of domain specific DNA nicking are followed or interspersed with one or more rounds domain specific evolution employing a plurality targeting polynucleotides described above.

The methods of the present invention also avoid multiple subcloning steps. This is particularly relevent when large complex vectors such as lambda, BACS, PACS, YACS, MACS and other genomic DNAs are used and where multiple subcloning steps make mutagenesis and shuffling of unique sites in large vectors particularly tedious and time consuming.

Accordingly, the present invention provides methods to introduce recombinogenic probe or hybrid complexes into recombination proficient cells to link in vitro and in vivo recombination and evolution processes. By generating homologous recombination intermediates in vitro, panels or libraries of mutagenized and shuffled genes are generated for in vitro envolution. The link to in vivo systems allows in vivo selection of evolved genes encoding proteins of a desired characteristic.

The present invention can thus be used in a variety of important ways. First, these methods can be used in the creation of transgenic organisms, animal, and plant models of disease. Thus, for example, domain-specific targeting polynucleotides used in homologous recombination methods can generate animals that have a wide variety of mutations in a wide variety of functionally related genes, potentially resulting in a wide variety of phenotypes, including phenotypes related to disease states. This may also be done on a cellular level, to identify genes involved in cellular phenotypes, i.e. target identification. Secondly, domain targeting can be used in cells or animals that are diseased or altered; in essence, domain targeting can be done to identify “reversion” genes, genes that can modulate disease states caused by different genes, either genes within the same gene family or a completely different gene family. Thus, for example the loss of one type of enzymatic activity, resulting in a disease phenotype, may be compensated by alterations in a different but homologous enzymatic activity.

In addition, the methods may be used in the creation of libraries of altered nucleic acids, including extrachromosomal sequences, and can be expressed in cells to produce libraries of altered proteins, which then can be screened for any number of useful or interesting properties, including, but not limited to, increased or altered stability (thermal, pH, oxidants, to proteases, etc.); altered specificity (for example, in the case of enzymes); altered binding; modified activity and other desirable properties, such as, altered immunogenicity.

Accordingly, the present invention provides methods of homologous recombination. By “homologous recombination” (HR) herein is meant an exchange of homologous or similar DNA sequence between two DNA molecules. An essential feature of HR is that the enzyme responsible for the recombination event can pair any homologous sequences as substrates. The ability of HR to transfer genetic information between DNA molecules makes targeted homologous recombination a very powerful method in genetic engineering and gene manipulation. HR can be used to insert, delete, and/or substitute any one or more nucleotides in a gene or gene segment or to introduce or delete genes in a targeted nucleic acid.

Once having identified a protein domain, the compositions of the invention can be made. The compositions of the invention comprise at least one recombinase and at least two single-stranded targeting polynucleotides which are substantially complementary to each other and each have a domain homology clamp.

By “recombinase” herein is meant a protein or peptide (e.g. L2 peptide) that, when included with an exogenous targeting polynucleotide, provide a measurable increase in the recombination frequency and/or localization frequency between the targeting polynucleotide and an endogenous predetermined DNA sequence. Thus, in a preferred embodiment, increases in recombination frequency from the normal range of 10⁻⁸ to 10⁻⁴, to 10⁻⁴ to 10¹, preferably 10⁻³ to 10¹, and most preferably 10⁻² to 10⁰, may be achieved.

In the present invention, recombinase refers to a family of RecA-like and Rad51-like recombination proteins all having essentially all or most of the same functions, particularly: (i) the recombinase protein's ability to properly bind to and position targeting polynucleotides on their homologous targets and (ii) the ability of recombinase protein/targeting polynucleotide complexes to efficiently find and bind to complementary endogenous sequences. The best characterized RecA protein is from E. coli, in addition to the wild-type protein a number of mutant RecA proteins have been identified (e.g., RecA803; see Madiraju et al., PNAS USA 85(18):6592 (1988); Madiraju et al, Biochem. 31:10529 (1992); Lavery et al., J. Biol. Chem. 267:20648 (1992)). Further, many organisms have RecA-like recombinases with strand-transfer activities (e.g., Fugisawa et al., (1985) Nucl. Acids Res. 13: 7473; Hsieh et al., (1986) Cell 44: 885; Hsieh et al., (1989) J. Biol. Chem. 264: 5089; Fishel et al., (1988) Proc. Natl. Acad. Sci. (USA) 85: 3683; Cassuto et al., (1987) Mol. Gen. Genet. 208: 10; Ganea et al., (1987) Mol. Cell Biol. 7: 3124; Moore et al., (1990) J. Biol. Chem. 19: 11108; Keene et al., (1984) Nucl. Acids Res. 12: 3057; Kimeic, (1984) Cold Spring Harbor Symp. 48: 675; Kmeic, (1986) Cell 44: 545; Kolodner et al., (1987) Proc. Natl. Acad. Sci. USA 84: 5560; Sugino et al., (1985) Proc. Natl. Acad. Sci. USA 85: 3683; Halbrook et al., (1989) J. Biol. Chem. 264: 21403; Eisen et al., (1988) Proc. Nati. Acad. Sci. USA 85: 7481; McCarthy et al., (1988) Proc. Natl. Acad. Sci. USA 85: 5854; Lowenhaupt et al., (1989) J. Biol. Chem. 264: 20568, which are incorporated herein by reference. Examples of such recombinase proteins include, for example but not limited to: RecA, RecA803, uvsX, and other RecA mutants and RecA-like recombinases (Roca, A. I. (1990) Crit. Rev. Biochem. Molec. Biol. 25: 415), sep1 (Kolodner et al. (1987) Proc. Natl. Acad. Sci. (U.S.A.) 84:5560; Tishkoff et al. Molec. Cell. Biol. 11:2593), RuvC (Dunderdale et al. (1991) Nature 354: 506), DST2, KEM1, XRN1 (Dykstra et al. (1991) Molec. Cell. Biol. 11:2583), STPα/DST1 (Clark et al. (1991) Molec. Cell. Biol. 11:2576), HPP-1 (Moore et al. (1991) Proc. Natl. Acad. Sci. (U.S.A.) 88:9067), other target recombinases (Bishop et al. (1992) Cell 69: 439; Shinohara et al. (1992) Cell 69: 457); incorporated herein by reference. RecA may be purified from E. coli strains, such as E. coli strains JC12772 and JC15369 (available from A. J. Clark and M. Madiraju, University of California-Berkeley, or purchased commercially). These strains contain the RecA coding sequences on a “runaway” replicating plasmid vector present at a high copy numbers per cell. The RecA803 protein is a high-activity mutant of wild-type RecA. The art teaches several examples of recombinase proteins, for example, from Drosophila, yeast, plant, human, and non-human mammalian cells, including proteins with biological properties similar to RecA (i.e., RecA-like recombinases), such as Rad51, Rad55, Rad57, dmcl from mammals and yeast. In addition, the recombinase may actually be a complex of proteins, i.e. a “recombinosome”. In addition, included within the definition of a recombinase are portions or fragments of recombinases which retain recombinase biological activity, as well as variants or mutants of wild-type recombinases which retain biological activity, such as the E. coli RecA803 mutant with enhanced recombinase activity.

In a preferred embodiment, RecA or rad51 is used. For example, RecA protein is typically obtained from bacterial strains that overproduce the protein: wild-type E. coli RecA protein and mutant RecA803 protein may be purified from such strains. Alternatively, RecA protein can also be purchased from, for example, Pharmacia (Piscataway, N.J.) or Boehringer Mannheim (Indianapolis, Ind.).

RecA proteins, and its homologs, form a nucleoprotein filament when it coats a single-stranded DNA. In this nucleoprotein filament, one monomer of RecA protein is bound to about 3 nucleotides. This property of RecA to coat single-stranded DNA is essentially sequence independent, although particular sequences favor initial loading of RecA onto a polynucleotide (e.g., nucleation sequences).

The nucleoprotein filament(s) can be formed on essentially any DNA molecule and can be formed in cells (e.g., mammalian cells), forming complexes with both single-stranded and double-stranded DNA, although the loading conditions for dsDNA are somewhat different than for ssDNA.

The recombinase is combined with targeting polynucleotides as is more fully outlined below. By “nucleic acid” or “oligonucleotide” or “polynucleotide” or grammatical equivalents herein means at least two nucleotides covalently linked together. A nucleic acid of the present invention will generally contain phosphodiester bonds, although in some cases nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate, phosphorodithioate, O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). These modifications of the ribose-phosphate backbone or bases may be done to facilitate the addition of other moieties such as chemical constituents, including 2′ O-methyl and 5′ modified substituents, as discussed below, or to increase the stability and half-life of such molecules in physiological environments.

The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo-and ribo-nucleotides, and any combination of bases, including uracil, adenine, thymine, cytosine, guanine, inosine, xathanine and hypoxathanine, etc. Thus, for example, chimeric DNA-RNA molecules may be used such as described in Cole-Strauss et al., Science 273:1386 (1996) and Yoon et al., PNAS USA 93:2071 (1996), both of which are hereby incorporated by reference.

In general, the targeting polynucleotides may comprise any number of structures, as long as the changes do not substantially effect the functional ability of the targeting polynucleotide to result in homologous recombination. For example, recombinase coating of alternate structures should still be able to occur.

By “targeting polynucleotides” herein is meant the polynucleotides used to make alterations in the protein domains as described herein. Targeting polynucleotides are generally ssDNA or dsDNA, most preferably two complementary single-stranded DNAs.

Targeting polynucleotides are generally at least about 5 to 2000 nucleotides long, preferably about 12 to 200 nucleotides long, at least about 200 to 500 nucleotides long, more preferably at least about 500 to 2000 nucleotides long, or longer; however, as the length of a targeting polynucleotide increases beyond about 20,000 to 50,000 to 400,000 nucleotides, the efficiency or transferring an intact targeting polynucleotide into the cell decreases. The length of homology may be selected at the discretion of the practitioner on the basis of the sequence composition and complexity of the predetermined endogenous target DNA sequence(s) and guidance provided in the art, which generally indicates that 1.3 to 6.8 kilobase segments of homology are preferred when non-recombinase mediated methods are utilized (Hasty et al. (1991) Molec. Cell. Biol. 11: 5586; Shulman et al. (1990) Molec. Cell. Biol. 10: 4466, which are incorporated herein by reference).

Targeting polynucleotides have at least one sequence that substantially corresponds to, or is substantially complementary to, a predetermined endogenous DNA sequence. As used herein, the terms “predetermined target nucleic acid” and “predetermined target sequence” and “predetermined domain of a target nucleic acid” refer to polynucleotide sequences contained in a target nucleic acid. Such sequences include, for example, chromosomal sequences (e.g., structural genes, regulatory sequences including promoters and enhancers, recombinatorial hotspots, repeat sequences, integrated proviral sequences, hairpins, palindromes), episomal or extrachromosomal sequences (e.g., replicable plasmids or viral replication intermediates) including chloroplast and mitochondrial DNA sequences. By “predetermined” or “pre-selected” it is meant that the target sequence may be selected at the discretion of the practitioner on the basis of known or predicted sequence information, and is not constrained to specific sites recognized by certain site-specific recombinases (e.g., FLP recombinase or CRE recombinase). In some embodiments, the predetermined endogenous DNA target sequence will be other than a naturally occurring germline DNA sequence (e.g., a transgene, parasitic, mycoplasmal or viral sequence). An exogenous polynucleotide is a polynucleotide which is transferred into a target cell but which has not been replicated in that host cell; for example, a virus genome polynucleotide that enters a cell by fusion of a virion to the cell is an exogenous polynucleotide, however, replicated copies of the viral polynucleotide subsequently made in the infected cell are endogenous sequences (and may, for example, become integrated into a cell chromosome). Similarly, transgenes which are microinjected or transfected into a cell are exogenous polynucleotides, however integrated and replicated copies of the transgene(s) are endogenous sequences.

In a preferred embodiment, the target nucleic acid comprises a nucleotide sequence encoding a protein or polypeptide, although as outlined herein, target nucleic acids may be made to non-coding regions as well. By “protein” herein is meant at least two covalently attached amino acids, which includes proteins, polypeptides, oligopeptides and peptides. Thus “amino acid” or “peptide residue”, as used herein means naturally occurring and naturally modified amino acids. For example, “amino acid” also includes imino acid residues such as proline and hydroxyproline. A “naturally modified amino acid” includes for examples, amino acids that are modified to contain carbohydrate structures, such as high-mannose or complex carbohydrates, phosphate, or lipids. In the preferred embodiment, the amino acids are in the (S) or L-configuration.

The nucleotide sequence encoding the polypeptide is preferably operably linked to transcription and translation control elements operable in a host cell of interest, such that, introduction of the target nucleic acid results in expression of the encoded protein. The transcription control elements include a promoter, such as, a constitutive or inducible promoter. When the host cell of interest is a eukaryotic cell, enhancer elements are optionally employed. In a preferred embodiment the target nucleic acid is an extrachromosomal vector such as a plasmid. In other embodiments, the target nucleic acid is a viral vector, such as, a retrovirus, a phage, a BAC, PAC, YAC, MAC or other types of genomic and chromosomal DNA.

The term “naturally-occurring” as used herein as applied to an object refers to the fact that an object can be found in nature. For example, a polynucleotide sequence that is present in an organism (including viruses) that can be isolated from a source in nature and which has not been intentionally modified by man in the laboratory is naturally-occurring.

The methods of the invention are used for alteration and evolution of protein domains; that is, in a preferred embodiment, the target nucleic acid comprises a nucleic acid encoding a protein domain. By “protein domain” and grammatical equivalents as used herein are meant a region of a protein that provides a specific structural and/or functional characteristic. Accordingly, a protein domain is an enzymatic active site, a ligand binding site, an allosteric effector region, an epitope, a region of a protein that is modified, such as, by addition of a carbohydrate, phosphate or lipid. A domain also relates to the hydrophobicity or hydrophilicity of a region and, therefore, also includes extracellular, intracellular, and transmembrane domains. Cell targeting sequences, such as, a signal peptide, nuclear localization sequence, mitochondrial localization sequences, etc. that direct proteins to either an extracellular or subcellular locale are domains. Additional domains include regions of proteins that interact with other proteins or nucleic acids, for example, include multimerization sequences, zinc-finger motifs, and the like. In another aspect, a protein domain is a region encoded by an exon.

Targeting polynucleotides have at least one sequence that substantially corresponds to, or is substantially complementary to, a target nucleic acid; in a preferred embodiment, it corresponds or complements a nucleic acid encoding a protein domain. By “corresponds to” herein is meant that a polynucleotide sequence is homologous (i.e., may be similar or identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence can hybridize to all or a portion of a reference polynucleotide sequence. Thus, one of the complementary single stranded targeting polynucleotides is complementary to one strand of the endogenous target domain sequence (i.e. Watson) and corresponds to the other strand of the endogenous target domain sequence (i.e. Crick). Thus, the complementarity between two single-stranded targeting polynucleotides need not be perfect. For illustration, the nucleotide sequence “TATAC” corresponds to a reference sequence “TATAC” and is perfectly complementary to a reference sequence “GTATA”.

The terms “substantially corresponds to” or “substantial identity” or “homologous” as used herein denotes a characteristic of a nucleic acid sequence, wherein a nucleic acid sequence has at least about 50 percent sequence identity as compared to a reference sequence, typically at least about 70 percent sequence identity, and preferably at least about 85 percent sequence identity as compared to a reference sequence. The percentage of sequence identity is calculated excluding small deletions or additions which total less than 25 percent of the reference sequence. The reference sequence may be a subset of a larger sequence, such as a portion of a gene or flanking sequence, or a repetitive portion of a chromosome. However, the reference sequence is at least 18 nucleotides long, typically at least about 30 nucleotides long, and preferably at least about 50 to 100 nucleotides long. “Substantially complementary” as used herein refers to a sequence that is complementary to a sequence that substantially corresponds to a reference sequence. In general, targeting efficiency increases with the length of the targeting polynucleotide portion that is substantially complementary to a reference sequence present in the target DNA.

By “sequence homology” herein is meant sequence similarity or sequence identity.

Nucleic acid similarity can be determined using, for example, BLASTN (Altschul et al. 1990. J. Mol. Biol. 147:195-197). BLASTN uses a simple scoring system in which matches count +5 and mismatches −4. To achieve computational efficiency, the default parameters have been incorporated directly into the source code.

In an alternative embodiment, percent nucleic acid sequence identity is determined. In percent identity calculations relative weight is not assigned to the various types of sequence variation, such as, insertions, deletions, substitutions, etc. Only identities are scored positively (+1) and all forms of sequence variation given a value of “0”, which obviates the need for a weighted scale or parameters as described above for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

These corresponding/complementary sequences are sometimes referred to herein as “domain homology clamps”, as they serve as templates for homologous pairing with the predetermined endogenous sequence(s). Thus, a “domain homology clamp” is a portion of the targeting polynucleotide that can specifically hybridize to a nucleic acid encoding a domain within a gene of interest. “Specific hybridization” is defined herein as the formation of hybrids between a targeting polynucleotide (e.g., a polynucleotide of the invention which may include substitutions, deletion, and/or additions as compared to the predetermined target nucleic acid sequence) and a predetermined target nucleic acid, wherein the targeting polynucleotide preferentially hybridizes to the predetermined target nucleic acid such that, for example, at least one discrete band can be identified on a Southern blot of nucleic acid prepared from target cells that contain the target nucleic acid sequence, and/or a targeting polynucleotide in an intact nucleus localizes to a discrete chromosomal location characteristic of a unique or repetitive sequence. As will be appreciated by those in the art, a target domain sequence may be present in more than one target polynucleotide species (e.g., a particular target sequence may occur in multiple members of a gene family). It is evident that optimal hybridization conditions will vary depending upon the sequence composition and length(s) of the targeting polynucleotide(s) and target(s), and the experimental method selected by the practitioner. Various guidelines may be used to select appropriate hybridization conditions (see, Maniatis et al., Molecular Cloning: A Laboratory Manual (1989), 2nd Ed., Cold Spring Harbor, N.Y. and Berger and Kimme1, Methods in Enzymology, Volume 152, Guide to Molecular Cloning Techniques (1987), Academic Press, Inc., San Diego, Calif.), which are incorporated herein by reference. Methods for hybridizing a targeting polynucleotide to a discrete chromosomal location in intact nuclei are known in the art, see for example WO 93/05177 and Kowalczykowski and Zarling (1994) in Gene Targeting, Ed. Manuel Vega.

In targeting polynucleotides, domain homology clamps are typically located at or near the 5′ or 3′ end, preferably domain homology clamps are internal or located at each end of the polynucleotide (Berinstein et al. (1992) Molec. Cell. Biol. 12: 360, which is incorporated herein by reference). Without wishing to be bound by any particular theory, it is believed that the addition of recombinases permits efficient gene targeting with targeting polynucleotides having short (i.e., about 10 to 1000 basepair long) segments of homology, as well as with targeting polynucleotides having longer segments of homology.

Therefore, it is preferred that targeting polynucleotides of the invention have domain homology clamps that are highly homologous to the predetermined target endogenous domain functional domain nucleic acid sequence(s). Typically, targeting polynucleotides of the invention have at least one domain homology clamp that is at least about 18 to 35 nucleotides long, and it is preferable that domain homology clamps are at least about 20 to 100 nucleotides long, and more preferably at least about 100-500 nucleotides long, although the degree of sequence homology between the domain homology clamp and the targeted sequence and the base composition of the targeted sequence will determine the optimal and minimal clamp lengths (e.g., G-C rich sequences are typically more thermodynamically stable and will generally require shorter clamp length). Therefore, both domain homology clamp length and the degree of sequence homology can only be determined with reference to a particular predetermined sequence, but domain homology clamps generally must be at least about 10 nucleotides long and must also substantially correspond or be substantially complementary to a predetermined target sequence. Preferably, a homology clamp is at least about 10, and preferably at least about 50 nucleotides long and is substantially identical to or complementary to a predetermined target sequence. Without wishing to be bound by a particular theory, it is believed that the addition of recombinases to a targeting polynucleotide enhances the efficiency of homologous recombination between homologous, nonisogenic sequences (e.g., between an exon 2 sequence of an albumin gene of a Balb/c mouse and a homologous albumin gene exon 2 sequence of a C57/BL6 mouse), as well as between isogenic sequences.

In one aspect of the invention, the targeting polynucleotides comprise a plurality of targeting polynucleotides comprising at least one shared homology clamp and a degenerate sequence. By “plurality” herein is meant more than one. The targeting polynucleotides find use in the mutagenesis and evolution of a target nucleic acid sequence that encodes specific protein domain by insertion, deletion and/or substitution of the nucleic acid sequence encoding the domain. In one embodiment the degenerate sequence is completely randomized, representing all possible combinations of nucleotides. In another embodiment, the degenerate sequence is biased, for example, to eliminate sequences encoding for transcriptional or translational stop signals. In another embodiment, the degenerate sequence is biased, to represent the codon bias of a host cell or class of organisms. The degenerate sequence is optionally biased to randomize specific sequence while maintaining other sequences constant. The length of the degenerate sequence is determined by the practitioner and is based on the desired number of nucleotides within the predetermined sequence to be modified.

In an alternative embodiment, the targeting polynucleotides are substantially identical to the predetermined target sequence. In the presence of a recombinase, the targeting polynucleotides form complexes with a predetermined target sequence of a target nucleic acid. As a part of the complex, the predetermined target sequence is resistant to nuclease digestion. The regions flanking the polynucleotide:target complex are susceptible to single-strand specific exonucleases. Accordingly, to effect domain specific evolution, these regions are nicked and the resultant fragments are reassembled and recombined by PCR as described below and by Stemmer et al. Nature. 370:389-391 and Stemmer et al. PNAS USA 91:10747-10751, hereby incorporated by reference.

The formation of heteroduplex joints is not a stringent process; genetic evidence supports the view that the classical phenomena of meiotic gene conversion and aberrant meiotic segregation results in part from the inclusion of mismatched base pairs in heteroduplex joints, and the subsequent correction of some of these mismatched base pairs before replication. Observations on RecA protein have provided information on parameters that affect the discrimination of relatedness from perfect or near-perfect homology and that affect the inclusion of mismatched base pairs in heteroduplex joints. The ability of RecA protein to drive strand exchange past all single base-pair mismatches and to form extensively mismatched joints in superhelical DNA reflect its role in recombination and gene conversion. This error-prone process may also be related to its role in mutagenesis. RecA-mediated pairing reactions involving DNA of φ×174 and G4, which are about 70 percent homologous, have yielded homologous recombinants (Cunningham et al. (1981) Cell 24: 213), although RecA preferentially forms homologous joints between highly homologous sequences, and is implicated as mediating a homology search process between an invading DNA strand and a recipient DNA strand, producing relatively stable heteroduplexes at regions of high homology. Accordingly, it is the fact that recombinases can drive the homologous recombination reaction between strands which are significantly, but not perfectly, homologous, which allows gene conversion and the modification of target sequences. Thus, targeting polynucleotides may be used to introduce nucleotide substitutions, insertions and deletions into an endogenous functional domain nucleic acid sequence, and thus the corresponding amino acid substitutions, insertions and deletions in proteins expressed from the endogenous domain functional domain nucleic acid sequence. By “endogenous” in this context herein is meant the naturally occurring sequence, i.e. sequences or substances originating from within a cell or organism. Similarly, “exogenous” refers to sequences or substances originating outside the cell or organism.

In a preferred embodiment, two substantially complementary targeting polynucleotides are used. In one embodiment, the targeting polynucleotides form a double stranded hybrid, which may be coated with recombinase, although when the recombinase is RecA, the loading conditions may be somewhat different from those used for single stranded nucleic acids.

In a preferred embodiment, two substantially complementary single-stranded targeting polynucleotides are used. The two complementary single-stranded targeting polynucleotides are usually of equal length, although this is not required. However, as noted below, the stability of the four strand hybrids of the invention is putatively related, in part, to the lack of significant unhybridized single-stranded nucleic acid, and thus significant unpaired sequences are not preferred. Furthermore, as noted above, the complementarity between the two targeting polynucleotides need not be perfect. The two complementary single-stranded targeting polynucleotides are simultaneously or contemporaneously introduced into a target cell harboring a predetermined endogenous target sequence, generally with at lease one recombinase protein (e.g., RecA). Under most circumstances, it is preferred that the targeting polynucleotides are incubated with RecA or other recombinase prior to introduction into a target cell, so that the recombinase protein(s) may be “loaded” onto the targeting polynucleotide(s), to coat the nucleic acid, as is described below. Incubation conditions for such recombinase loading are described infra, and also in U.S. Ser. No. 07/755,462, filed Sep. 4, 1991; U.S. Ser. No. 07/910,791, filed Jul. 9, 1992; and U.S. Ser. No. 07/520,321, filed May 7, 1990, each of which is incorporated herein by reference. A targeting polynucleotide may contain a sequence that enhances the loading process of a recombinase, for example a RecA loading sequence is the recombinogenic nucleation sequence poly[d(A-C)], and its complement, poly[d(G-T)]. The duplex sequence poly[d(A-C)·d(G-T)ₙ, where n is from 5 to 25, is a middle repetitive element in target DNA.

There appears to be a fundamental difference in the stability of RecA-protein-mediated D-loops formed between one single-stranded DNA (ssDNA) probe hybridized to negatively supercoiled DNA targets in comparison to relaxed or linear duplex DNA targets. Internally located dsDNA target sequences on relaxed linear DNA targets hybridized by ssDNA probes produce single D-loops, which are unstable after removal of RecA protein (Adzuma, Genes Devel. 6:1679 (1992); Hsieh et al, PNAS USA 89:6492 (1992); Chiu et al., Biochemistry 32:13146 (1993)). This probe DNA instability of hybrids formed with linear duplex DNA targets is most probably due to the incoming ssDNA probe W-C base pairing with the complementary DNA strand of the duplex target and disrupting the base pairing in the other DNA strand. The required high free-energy of maintaining a disrupted DNA strand in an unpaired ssDNA conformation in a protein-free single-D-loop apparently can only be compensated for either by the stored free energy inherent in negatively supercoiled DNA targets or by base pairing initiated at the distal ends of the joint DNA molecule, allowing the exchanged strands to freely intertwine.

However, the addition of a second complementary ssDNA to the three-strand-containing single-D-loop stabilizes the deproteinized hybrid joint molecules by allowing W-C base pairing of the probe with the displaced target DNA strand. The addition of a second RecA-coated complementary ssDNA (cssDNA) strand to the three-strand containing single D-loop stabilizes deproteinized hybrid joints located away from the free ends of the duplex target DNA (Sena & Zarling, Nature Genetics 3:365 (1993); Revet et al. J. Mol. Biol. 232:779 (1993); Jayasena and Johnston, J. Mol. Bio. 230:1015 (1993)). The resulting four-stranded structure, named a double D-loop by analogy with the three-stranded single D-loop hybrid has been shown to be stable in the absence of RecA protein. This stability likely occurs because the restoration of W-C basepairing in the parental duplex would require disruption of two W-C basepairs in the double-D-loop (one W-C pair in each heteroduplex D-loop). Since each base-pairing in the reverse transition (double-D-loop to duplex) is less favorable by the energy of one W-C basepair, the pair of cssDNA probes are thus kinetically trapped in duplex DNA targets in stable hybrid structures. The stability of the double-D loop joint molecule within internally located probe:target hybrids is an intermediate stage prior to the progression of the homologous recombination reaction to the strand exchange phase. The double D-loop permits isolation of stable multistranded DNA recombination intermediates.

In addition, when the targeting polynucleotides are used to generate insertions or deletions in an endogenous nucleic acid sequence, as is described herein, the use of two complementary single-stranded targeting polynucleotides allows the use of internal homology clamps as depicted in the figures of PCT US98/05223. The use of internal homology clamps allows the formation of stable deproteinized cssDNA:probe target hybrids with homologous DNA sequences containing either relatively small or large insertions and deletions within a homologous DNA target. Without being bound by theory, it appears that these probe:target hybrids, with heterologous inserts in the cssDNA probe, are stabilized by the re-annealing of cssDNA probes to each other within the double-D-loop hybrid, forming a novel DNA structure with an internal homology clamp. Similarly stable double-D-loop hybrids formed at internal sites with heterologous inserts in the linear DNA targets (with respect to the cssDNA probe) are equally stable. Because cssDNA probes are kinetically trapped within the duplex target, the multi-stranded DNA intermediates of homologous DNA pairing are stabilized and strand exchange is facilitated.

In a preferred embodiment, the length of the internal homology clamp (i.e. the length of the insertion or deletion) is from about 1 to 50% of the total length of the targeting polynucleotide, with from about 1 to about 20% being preferred and from about 1 to about 10% being especially preferred, although in some cases the length of the deletion or insertion may be significantly larger. As for the domain homology clamps, the complementarity within the internal homology clamp need not be perfect.

A targeting polynucleotide used in a method of the invention typically is a single-stranded nucleic acid, usually a DNA strand, or derived by denaturation of a duplex DNA, which is complementary to one (or both) strand(s) of the target duplex nucleic acid. Thus, one of the complementary single stranded targeting polynucleotides is complementary to one strand of the endogenous target sequence (i.e. Watson) and the other complementary single stranded targeting polynucleotide is complementary to the other strand of the endogenous target sequence (i.e. Crick). The domain homology clamp sequence preferably contains at least 90-95% sequence homology with the target sequence (although as outlined above, less sequence homology can be tolerated), to insure sequence-specific targeting of the targeting polynucleotide to the endogenous DNA domain target. Each single-stranded targeting polynucleotide is typically about 50-600 bases long, although a shorter or longer polynucleotide may also be employed.

Once the gene family and domain sequence is selected, the targeting polynucleotides are made, as will be appreciated by those in the art. For example, for large targeting polynucleotides, plasmids are engineered to contain an appropriately sized gene sequence with a deletion or insertion in the gene of interest and at least one flanking homology clamp which substantially corresponds or is substantially complementary to an endogenous target DNA sequence. Vectors containing a targeting polynucleotide sequence are typically grown in E. coli and then isolated using standard molecular biology methods. Alternatively, targeting polynucleotides may be prepared in single-stranded form by oligonucleotide synthesis methods, which may first require, especially with larger targeting polynucleotides, formation of subfragments of the targeting polynucleotide, typically followed by splicing of the subfragments together, typically by enzymatic ligation. In general, as will be appreciated by those in the art, targeting polynucleotides may be produced by chemical synthesis of oligonucleotides, nick-translation of a double-stranded DNA template, polymerase chain-reaction amplification of a sequence (or ligase chain reaction amplification), purification of prokaryotic or target cloning vectors harboring a sequence of interest (e.g., a cloned cDNA or genomic clone, or portion thereof) such as plasmids, phagemids, YACs, cosmids, bacteriophage DNA, other viral DNA or replication intermediates, or purified restriction fragments thereof, as well as other sources of single and double-stranded polynucleotides having a desired nucleotide sequence. When using microinjection procedures it may be preferable to use a transfection technique with linearized sequences containing only modified target gene sequence and without vector or selectable sequences. The modified gene site is such that a homologous recombinant between the exogenous targeting polynucleotide and the endogenous DNA target sequence can be identified by using carefully chosen primers and PCR, followed by analysis to detect if PCR products specific to the desired targeted event are present (Erlich et al., (1991) Science 252: 1643, which is incorporated herein by reference). Several studies have already used PCR to successfully identify and then clone the desired transfected cell lines (Zimmer and Gruss, (1989) Nature 338: 150; Mouellic et al., (1990) Proc. Natl. Acad. Sci. USA 87: 4712; Shesely et al., (1991) Proc. Natl. Acad. Sci. USA 88: 4294, which are incorporated herein by reference). This approach is very effective when the number of cells receiving exogenous targeting polynucleotide(s) is high (i.e., with microinjection, or with liposomes) and the treated cell populations are allowed to expand to cell groups of approximately 1×10⁴ cells (Capecchi, (1989) Science 244: 1288). When the target gene is not on a sex chromosome, or the cells are derived from a female, both alleles of a gene can be targeted by sequential inactivation (Mortensen et al., (1991) Proc. Natl. Acad. Sci. USA 88: 7036). Alternatively, animals heterologous for the target gene can be bred to homologously as is known in the art.

The present invention allows for the introduction of alterations in the target nucleic acid comprising a domain or domains of interest. That is, the fact that heterologies are tolerated in targeting polynucleotides allows for two things: first, the use of a heterologous domain homology clamps that may target genes encoding functional domains of a protein or multiple proteins, resulting in a variety of genotypes and phenotypes, and secondly, the introduction of alterations to the target sequence. Thus typically, a targeting polynucleotide (or complementary polynucleotide pair) has a portion or region having a sequence that is not present in the preselected endogenous targeted sequence(s) (i.e., a nonhomologous portion or mismatch) which may be as small as a single mismatched nucleotide, several mismatches, or may span up to about several kilobases or more of nonhomologous sequence.

Accordingly, in a preferred embodiment, the methods and compositions of the invention are used for inactivation of a domain of a gene. That is, exogenous targeting polynucleotides can be used to inactivate, decrease or alter the biological activity of one or more domains in a gene of a cell (or transgenic nonhuman animal or plant). This finds particular use in the generation of animal models of disease states, or in the elucidation of gene function and activity, similar to “knock out” experiments. Alternatively, the biological activity of the wild-type gene may be either decreased, or the wild-type activity altered to mimic disease states. This includes genetic manipulation of non-coding gene sequences that affect the transcription of genes, including, promoters, repressors, enhancers and transcriptional activating sequences.

Thus in a preferred embodiment, homologous recombination of the targeting polynucleotide and endogenous target sequence will result in amino acid substitutions, insertions or deletions in the endogenous target sequences, potentially both within the functional domain region and outside of it, for example as a result of the incorporation of PCR tags. This will generally result in modulated or altered gene function of the endogenous gene, including both a decrease or elimination of function as well as an enhancement of function. Nonhomologous portions are used to make insertions, deletions, and/or replacements in a predetermined endogenous targeted DNA sequence, and/or to make single or multiple nucleotide substitutions in a predetermined endogenous target DNA sequence so that the resultant recombined sequence (i.e., a targeted recombinant endogenous sequence) incorporates some or all of the sequence information of the nonhomologous portion of the targeting polynucleotide(s). Thus, the nonhomologous regions are used to make variant sequences, i.e. targeted sequence modifications. In this way, site directed modifications may be done in a variety of systems for a variety of purposes.

The endogenous target sequence, generally nucleic acid encoding a domain, may be disrupted in a variety of ways. The term “disrupt” as used herein comprises a change in the coding or non-coding sequence of an endogenous nucleic acid. In one preferred embodiment, a disrupted gene will no longer produce a functional gene product. In another preferred embodiment, a disrupted gene produces a variant gene product. Generally, disruption may occur by either the substitution, insertion, deletion or frame shifting of nucleotides.

In one embodiment, amino acid substitutions are made. This can be the result of either the incorporation of a non-naturally occurring domain sequence into a target, or of more specific changes to a particular sequence outside of the domain sequence.

In one embodiment, the endogenous sequence is disrupted by an insertion sequence. The term “insertion sequence” as used herein means one or more nucleotides which are inserted into an endogenous gene to disrupt it. In general, insertion sequences can be as short as 1 nucleotide or as long as a gene, as outlined herein. For non-gene insertion sequences, the sequences are at least 1 nucleotide, with from about 1 to about 50 nucleotides being preferred, and from about 10 to 25 nucleotides being particularly preferred. An insertion sequence may comprise a polylinker sequence, with from about 1 to about 50 nucleotides being preferred, and from about 10 to 25 nucleotides being particularly preferred. Insertion sequence may be a PCR tag used for identification of the first gene.

In a preferred embodiment, an insertion sequence comprises a gene which not only disrupts the endogenous gene, thus preventing its expression, but also can result in the expression of a new gene product. Thus, in a preferred embodiment, the disruption of an endogenous gene by an insertion sequence gene is done in such a manner to allow the transcription and translation of the insertion gene. An insertion sequence that encodes a gene may range from about 50 bp to 5000 bp of cDNA or about 5000 bp to 50000 bp of genomic DNA. As will be appreciated by those in the art, this can be done in a variety of ways. In a preferred embodiment, the insertion gene is targeted to the endogenous gene in such a manner as to utilize endogenous regulatory sequences, including promoters, enhancers or a regulatory sequence. In an alternate embodiment, the insertion sequence gene includes its own regulatory sequences, such as a promoter, enhancer or other regulatory sequence etc.

Particularly preferred insertion sequence genes include, but are not limited to, genes which encode selection or reporter proteins. In addition, the insertion sequence genes may be modified or variant genes.

The term “deletion” as used herein comprises removal of a portion of the nucleic acid sequence of an endogenous gene. Deletions range from about 1 to about 100 nucleotides, with from about 1 to 50 nucleotides being preferred and from about 1 to about 25 nucleotides being particularly preferred, although in some cases deletions may be much larger, and may effectively comprise the removal of the entire functional domain, the entire endogenous gene and/or its regulatory sequences. Deletions may occur in combination with substitutions or modifications to arrive at a final modified endogenous gene.

In a preferred embodiment, endogenous genes may be disrupted simultaneously by an insertion and a deletion. For example, a domain of an endogenous gene, with or without its regulatory sequences, may be removed and replaced with an insertion sequence gene. Thus, for example, all but the regulatory sequences of an endogenous gene may be removed, and replaced with an insertion sequence gene, which is now under the control of the endogenous gene's regulatory elements.

The term “regulatory element” is used herein to describe a non-coding sequence which affects the transcription or translation of a gene including, but are not limited to, promoter sequences, ribosomal binding sites, transcriptional start and stop sequences, translational start and stop sequences, enhancer or activator sequences, dimerizing sequences, etc. In a preferred embodiment, the regulatory sequences include a promoter and transcriptional start and stop sequence. Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition to domain homology clamps and optional internal homology clamps, the targeting polynucleotides of the invention may comprise additional components, such as cell-uptake components, chemical substituents, purification tags, etc.

In a preferred embodiment, at least one of the targeting polynucleotides comprises at least one cell-uptake component. As used herein, the term “cell-uptake component” refers to an agent which, when bound, either directly or indirectly, to a targeting polynucleotide, enhances the intracellular uptake of the targeting polynucleotide into at least one cell type (e.g., hepatocytes). A targeting polynucleotide of the invention may optionally be conjugated, typically by covalently or preferably noncovalent binding, to a cell-uptake component. Various methods have been described in the art for targeting DNA to specific cell types. A targeting polynucleotide of the invention can be conjugated to essentially any of several cell-uptake components known in the art. For targeting to hepatocytes, a targeting polynucleotide can be conjugated to an asialoorosomucoid (ASOR)-poly-L-lysine conjugate by methods described in the art and incorporated herein by reference (Wu GY and Wu CH (1987) J. Biol. Chem. 262:4429; Wu GY and Wu CH (1988) Biochemistry 27:887; Wu GY and Wu CH (1988) J. Biol. Chem. 263: 14621; Wu GY and Wu CH (1992) J. Biol. Chem. 267:12436; Wu et al. (1991) J. Biol. Chem. 266: 14338; and Wilson et al. (1992) J. Biol. Chem. 267: 963, WO92/06180; WO92/05250; and WO91/17761, which are incorporated herein by reference).

Alternatively, a cell-uptake component may be formed by incubating the targeting polynucleotide with at least one lipid species and at least one protein species to form protein-lipid-polynucleotide complexes consisting essentially of the targeting polynucleotide and the lipid-protein cell-uptake component. Lipid vesicles made according to Felgner (WO91/17424, incorporated herein by reference) and/or cationic lipidization (WO91/16024, incorporated herein by reference) or other forms for polynucleotide administration (EP 465,529, incorporated herein by reference) may also be employed as cell-uptake components. Nucleases, DNA damaging chemicals, UV radiation or gamma-radiation may also be used.

In addition to cell-uptake components, targeting components such as nuclear localization signals may be used, as is known in the art. See for example Kido et al., Exper. Cell Res. 198:107-114 (1992), hereby expressly incorporated by reference.

Typically, a targeting polynucleotide of the invention is coated with at least one recombinase and is conjugated to a cell-uptake component, and the resulting cell targeting complex is contacted with a target cell under uptake conditions (e.g., physiological conditions) so that the targeting polynucleotide and the recombinase(s) are internalized in the target cell. A targeting polynucleotide may be contacted simultaneously or sequentially with a cell-uptake component and also with a recombinase; preferably the targeting polynucleotide is contacted first with a recombinase, or with a mixture comprising both a cell-uptake component and a recombinase under conditions whereby, on average, at least about one molecule of recombinase is noncovalently attached per targeting polynucleotide molecule and at least about one cell-uptake component also is noncovalently attached. Most preferably, coating of both recombinase and cell-uptake component saturates essentially all of the available binding sites on the targeting polynucleotide. A targeting polynucleotide may be preferentially coated with a cell-uptake component so that the resultant targeting complex comprises, on a molar basis, more cell-uptake component than recombinase(s). Alternatively, a targeting polynucleotide may be preferentially coated with recombinase(s) so that the resultant targeting complex comprises, on a molar basis, more recombinase(s) than cell-uptake component.

Cell-uptake components are included with recombinase-coated targeting polynucleotides of the invention to enhance the uptake of the recombinase-coated targeting polynucleotide(s) into cells, particularly for in vivo gene targeting applications, such as gene therapy to treat genetic diseases, including neoplasia, and targeted homologous recombination to treat viral infections wherein a viral sequence (e.g., an integrated hepatitis B virus (HBV) genome or genome fragment) may be targeted by homologous sequence targeting and inactivated. Alternatively, a targeting polynucleotide may be coated with the cell-uptake component and targeted to cells with a contemporaneous or simultaneous administration of a recombinase (e.g., liposomes or immunoliposomes containing a recombinase, a viral-based vector encoding and expressing a recombinase).

In addition to recombinase and cellular uptake components, at least one of the targeting polynucleotides may include chemical substituents. Exogenous targeting polynucleotides that have been modified with appended chemical substituents may be introduced along with recombinase (e.g., RecA) into a metabolically active target cell to homologously pair with a predetermined endogenous DNA target sequence in the cell. In a preferred embodiment, the exogenous targeting polynucleotides are derivatized, and additional chemical substituents are attached, either during or after polynucleotide synthesis, respectively, and are thus localized to a specific endogenous target sequence where they produce an alteration or chemical modification to a local DNA sequence. Preferred attached chemical substituents include, but are not limited to: cross-linking agents (see Podyminogin et al., Biochem. 34:13098 (1995) and 35:7267 (1996), both of which are hereby incorporated by reference), nucleic acid cleavage agents, metal chelates (e.g., iron/EDTA chelate for iron catalyzed cleavage), topoisomerases, endonucleases, exonucleases, ligases, phosphodiesterases, photodynamic porphyrins, chemotherapeutic drugs (e.g., adriamycin, doxirubicin), intercalating agents, labels, base-modification agents, agents which normally bind to nucleic acids such as labels, etc. (see for example Afonina et al., PNAS USA 93:3199 (1996), incorporated herein by reference) immunoglobulin chains, and oligonucleotides. Iron/EDTA chelates are particularly preferred chemical substituents where local cleavage of a DNA sequence is desired (Hertzberg et al. (1982) J. Am. Chem. Soc. 104: 313; Hertzberg and Dervan (1984) Biochemistry 23: 3934; Taylor et al. (1984) Tetrahedron 40: 457; Dervan, PB (1986) Science 232: 464, which are incorporated herein by reference). Further preferred are groups that prevent hybridization of the complementary single stranded nucleic acids to each other but not to unmodified nucleic acids; see for example Kutryavin et al., Biochem. 35:11170 (1996) and Woo et al., Nucleic Acid. Res. 24(13):2470 (1996), both of which are incorporated by reference. 2′-O methyl groups are also preferred; see Cole-Strauss et al., Science 273:1386 (1996); Yoon et al., PNAS 93:2071 (1996)). Additional preferred chemical substituents include labeling moieties, including fluorescent labels. Preferred attachment chemistries include: direct linkage, e.g., via an appended reactive amino group (Corey and Schultz (1988) Science 238:1401, which is incorporated herein by reference) and other direct linkage chemistries, although streptavidin/biotin and digoxigenin/antidigoxigenin antibody linkage methods may also be used. Methods for linking chemical substituents are provided in U.S. Pat. Nos. 5,135,720, 5,093,245, and 5,055,556, which are incorporated herein by reference. Other linkage chemistries may be used at the discretion of the practitioner.

In a preferred embodiment, at least one of the targeting polynucleotides comprises at least one purification tag or capture moiety, some of which are discussed above as chemical substituents, for example biotin, digoxigenin, psoralen, etc. Alternatively, the domain oligonucleotide could be directly attached to beads with the targeting reaction performed on a solid phase support.

In a preferred embodiment, the targeting polynucleotides are coated with recombinase prior to introduction to the domain target. The conditions used to coat targeting polynucleotides with recombinases such as RecA protein and ATPγS have been described in commonly assigned U.S. Ser. No. 07/910,791, filed Jul. 9, 1992; U.S. Ser. No. 07/755,462, filed Sep. 4, 1991; and U.S. Ser. No. 07/520,321, filed May 7, 1990, and PCT US98/05223, each incorporated herein by reference. The procedures below are directed to the use of E. coli RecA, although as will be appreciated by those in the art, other recombinases may be used as well. Targeting polynucleotides can be coated using GTPγS, mixes of ATPγS with rATP, rGTP and/or dATP, or dATP or rATP alone in the presence of an rATP generating system (Boehringer Mannheim). Various mixtures of GTPγS, ATPγS, ATP, ADP, dATP and/or rATP or other nucleosides may be used, particularly preferred are mixes of ATPγS and ATP or ATPγS and ADP.

RecA protein coating of targeting polynucleotides is typically carried out as described in U.S. Ser. No . 07/910,791, filed Jul. 9, 1992 and U.S. Ser. No. 07/755,462, filed Sep. 4, 1991, and PCT US98/05223, which are incorporated herein by reference. Briefly, the targeting polynucleotide, whether double-stranded or single-stranded, is denatured by heating in an aqueous solution at 95-100° C. for five minutes, then placed in an ice bath for 20 seconds to about one minute followed by centrifugation at 0° C. for approximately 20 sec, before use. When denatured targeting polynucleotides are not placed in a freezer at −20° C. they are usually immediately added to standard RecA coating reaction buffer containing ATPγS, at room temperature, and to this is added the RecA protein. Alternatively, RecA protein may be included with the buffer components and ATPγS before the polynucleotides are added.

RecA coating of targeting polynucleotide(s) is initiated by incubating polynucleotide-RecA mixtures at 37° C. for 10-15 min. RecA protein concentration tested during reaction with polynucleotide varies depending upon polynucleotide size and the amount of added polynucleotide, and the ratio of RecA molecule:nucleotide preferably ranges between about 3:1 and 1:3. When single-stranded polynucleotides are RecA coated independently of their homologous polynucleotide strands, the mM and μM concentrations of ATPγS and RecA, respectively, can be reduced to one-half those used with double-stranded targeting polynucleotides (i.e., RecA and ATPγS concentration ratios are usually kept constant at a specific concentration of individual polynucleotide strand, depending on whether a single- or double-stranded polynucleotide is used).

RecA protein coating of targeting polynucleotides is normally carried out in a standard 1×RecA coating reaction buffer. 10× RecA reaction buffer (i.e., 10× AC buffer) consists of: 100 mM Tris acetate (pH 7.5 at 37° C.), 20 mM magnesium acetate, 500 mM sodium acetate, 10 mM DTT, and 50% glycerol). All of the targeting polynucleotides, whether double-stranded or single-stranded, typically are denatured before use by heating to 95-100° C. for five minutes, placed on ice for one minute, and subjected to centrifugation (10,000 rpm) at 0° C. for approximately 20 seconds (e.g., in a Tomy centrifuge). Denatured targeting polynucleotides usually are added immediately to room temperature RecA coating reaction buffer mixed with ATPγS and diluted with double-distilled H₂O as necessary.

A reaction mixture typically contains the following components: (i) 0.2-4.8 mM ATPγS; and (ii) between 1-100 ng/μl of targeting polynucleotide. To this mixture is added about 1-20 μl of RecA protein per 10-100 μl of reaction mixture, usually at about 2-10 mg/ml (purchased from Pharmacia or purified), and is rapidly added and mixed. The final reaction volume-for RecA coating of targeting polynucleotide is usually in the range of about 10-500 μl. RecA coating of targeting polynucleotide is usually initiated by incubating targeting polynucleotide-RecA mixtures at 37° C. for about 10-15 min.

RecA protein concentrations in coating reactions varies depending upon targeting polynucleotide size and the amount of added targeting polynucleotide: RecA protein concentrations are typically in the range of 5 to 50 μM. When single-stranded targeting polynucleotides are coated with RecA, independently of their complementary strands, the concentrations of ATPγS and RecA protein may optionally be reduced to about one-half of the concentrations used with double-stranded targeting polynucleotides of the same length: that is, the RecA protein and ATPγS concentration ratios are generally kept constant for a given concentration of individual polynucleotide strands.

The coating of targeting polynucleotides with RecA protein can be evaluated in a number of ways. First, protein binding to DNA can be examined using band-shift gel assays (McEntee et al., (1981) J. Biol. Chem. 256: 8835). Labeled polynucleotides can be coated with RecA protein in the presence of ATPγS and the products of the coating reactions may be separated by agarose gel electrophoresis. Following incubation of RecA protein with denatured duplex DNAs the RecA protein effectively coats single-stranded targeting polynucleotides derived from denaturing a duplex DNA. As the ratio of RecA protein monomers to nucleotides in the targeting polynucleotide increases from 0, 1:27, 1:2.7 to 3.7:1 for 121-mer and 0, 1:22, 1:2.2 to 4.5:1 for 159-mer, targeting polynucleotide's electrophoretic mobility decreases, i.e., is retarded, due to RecA-binding to the targeting polynucleotide. Retardation of the coated polynucleotide's mobility reflects the saturation of targeting polynucleotide with RecA protein. An excess of RecA monomers to DNA nucleotides is required for efficient RecA coating of short targeting polynucleotides (Leahy et al., (1986) J. Biol. Chem. 261: 954).

A second method for evaluating protein binding to DNA is in the use of nitrocellulose fiber binding assays (Leahy et al., (1986) J. Biol. Chem. 261:6954; Woodbury, et al., (1983) Biochemistry 22(20):4730-4737. The nitrocellulose filter binding method is particularly useful in determining the dissociation-rates for protein:DNA complexes using labeled DNA. In the filter binding assay, DNA:protein complexes are retained on a filter while free DNA passes through the filter. This assay method is more quantitative for dissociation-rate determinations because the separation of DNA:protein complexes from free targeting polynucleotide is very rapid.

Alternatively, recombinase protein(s) (prokaryotic, eukaryotic or endogeneous to the target cell) may be exogenously induced or administered to a target cell simultaneously or contemporaneously (i.e., within about a few hours) with the targeting polynucleotide(s). Such administration is typically done by micro-injection, although electroporation, lipofection, and other transfection methods known in the art may also be used. Alternatively, recombinase-proteins may be produced in vivo. For example, they may be produced from a homologous or heterologous expression cassette in a transfected cell or targeted cell, such as a transgenic totipotent cell (e.g. a fertilized zygote) or an embryonal stem cell (e.g., a murine ES cell such as AB-1) used to generate a transgenic non-human animal line or a somatic cell or a pluripotent hematopoietic stem cell for reconstituting all or part of a particular stem cell population (e.g. hematopoietic) of an individual. Conveniently, a heterologous expression cassette includes a modulatable promoter, such as an ecdysone-inducible promoter-enhancer combination, an estrogen-induced promoter-enhancer combination, a CMV promoter-enhancer, an insulin gene promoter, or other cell-type specific, developmental stage-specific, hormone-inducible drug inducible, or other modulatable promoter construct so that expression of at least one species of recombinase protein from the cassette can by modulated for transiently producing recombinase(s) in vivo simultaneous or contemporaneous with introduction of a targeting polynucleotide into the cell. When a hormone-inducible promoter-enhancer combination is used, the cell must have the required hormone receptor present, either naturally or as a consequence of expression a co-transfected expression vector encoding such receptor. Alternatively, the recombinase may be endogeneous and produced in high levels. In this embodiment, preferably in eukaryotic target cells such as tumor cells, the target cells produce an elevated level of recombinase. In other embodiments the level of recombinase may be induced by DNA damaging agents, such as mitomycin C, UV or γ-irradiation. Alternatively, recombinase levels may be elevated by transfection of a plasmid encoding the recombinase gene into the cell.

Once made, the compositions of the invention find use in a number of applications upon administration to target cells. In general, the compositions and methods of the invention are useful to identify new members of gene families which may be useful in functional genomic studies as well as in the identification of new drug targets; both of these may be accomplished through the generation of “knock out” animal models. In addition, the present invention allows the modification of functional domain targets, the creation of transgenic plants and animals, the cloning of genes containing domain functional domains, etc.

Once made and administered to a target host cell, the compositions of the invention find use in a number of applications, including domain specific gene evolution. The polypeptide or protein encoded by the targeted nucleic undegoes homologous recombination with the plurality of polynucleotides to produce a plurality of modified target nucleic acids that are expressed to produce a plurality of modified proteins. Selection systems are employed to identify and isolate host cells expressing proteins having a desired property or phenotype. For example, if the expressed protein is an enzyme, cells having a modified enzyme activity are identified. The desired activity can be an increased or decreased or altered activity. Proteins having the desired phenotype are selected and isolated, the modified nucleic acid is sequenced to identify sequences effecting the desired activity, and the process is repeated iteratively as needed to produce a protein having a desired activity or property.

In this and other embodiments, suitable target sequences include nucleic acid sequences encoding therapeutically or commercially relevant proteins, including, but not limited to, enzymes (proteases, recombinases, lipases, kinases, carbohydrases, isomerases, tautomerases, nucleases etc.), hormones, receptors, transcription factors, growth factors, cytokines, globin genes, immunosupppressive genes, tumor suppressors, oncogenes, complement-activating genes, milk proteins (casein, α-lactalbumin, β-lactoglobulin, bovine and human serum albumin), immunoglobulins, milk proteins, and pharmaceutical proteins and vaccines.

In a preferred embodiment, the methods of the invention are used to generate pools or libraries of variant nucleic acid sequences, and cellular libraries containing the variant sequences. This idea is somewhat similar to the “gene shuffling” techniques of the literature (see Stemmer et al., 1994, Natuere 370:389 which attempt to rapidly “evolve” genes by making multiple random changes simultaneously. In the present invention, this end is accomplished by using at least one cycle, and preferably reiterative cycles, of enhanced homologous recombination with targeting polynucleotides containing random mismatches, substitutions, insertions, or deletions. By using a library of targeting polynucleotides comprising a plurality of random mutations, and repeating the homologous recombination steps as many times as needed, a rapid “gene evolution” can occur, wherein the new genes may contain large numbers of mutations.

Thus, in this embodiment, a plurality of targeting polynucleotides are used. The targeting polynucleotides each have at least one homology clamp that substantially corresponds to or is substantially complementary to the target sequence. Generally, the targeting polynucleotides are generated in pairs; that is, pairs of two single stranded targeting polynucleotides that are substantially complementary to each other are made (i.e. a Watson strand and a Crick strand). However, as will be appreciated by those in the art, less than a one to one ratio of Watson to Crick strands may be used; for example, an excess of one of the single stranded target polynucleotides (i.e. Watson) may be used. Preferably, sufficient numbers of each of Watson and Crick strands are used to allow the majority of the targeting polynucleotides to form double D-loops, which are preferred over single D-loops as outlined above. In addition, the pairs need not have perfect complementarity; for example, an excess of one of the single stranded target polynucleotides (i.e. Watson), which may or may not contain mismatches, may be paired to a large number of variant Crick strands, etc. Due to the random nature of the pairing, one or both of any particular pair of single-stranded targeting polynucleotides may not contain any mismatches. However, generally, at least one of the strands will contain at least one mismatch.

The plurality of pairs preferably comprise a pool or library of mismatches. The size of the library will depend on a number of factors, including the number of residues to be mutagenized, the succeptibility of the protein to mutation, etc., as will be appreciated by those in the art. Generally, a library in this instance preferably comprises at least 10% different mismatches over the length of the targeting polynucleotides, with at least 30% mismatches being preferred and at least 40% being particularly preferred, although as will be appreciated by those in the art, lower (1, 2, 5%, etc.) or higher amounts of mismatches being both possible and desirable in some instances. That is, the plurality of pairs comprise a pool of random and preferably degenerate mismatches over some regions or all of the entire targeting sequence. As outlined herein, “mismatches” include substitutions, insertions and deletions, with the former being preferred. Thus, for example, a pool of degenerate variant targeting polynucleotides covering some, or preferably all, possible mismatches over some region are generated, as outlined above, using techniques well known in the art. Preferably, but not required, the variant targeting polynucleotides each comprise only one or a few mismatches (less than 10), to allow complete multiple randomization. That is, by repeating the homologous recombination steps any number of times, as is more fully outlined below, the mismatches from a plurality of probes can be incorporated into a single target sequence.

The mismatches can be either non-random (i.e. targeted) or random, including biased randomness. That is, in some instances specific changes are desirable, and thus the sequence of the targeting polynucleotides are specifically chosen. In a preferred embodiment, the mismatches are random. The targeting polynucleotides can be chemically synthesized, and thus may incorporate any nucleotide at any position. The synthetic process can be designed to generate randomized nucleic acids, to allow the formation of all or most of the possible combinations over the length of the nucleic acid, thus forming a library of randomized targeting polynucleotides. Preferred methods maximize library size and diversity.

It is important to understand that in any library system encoded by oligonucleotide synthesis one cannot have complete control over the codons that will eventually be incorporated into the peptide structure. This is especially true in the case of codons encoding stop signals (TAA, TGA, TAG). In a synthesis with NNN as the random region, there is a 3/64, or 4.69%, chance that the codon will be a stop codon. To alleviate this, random residues are encoded as NNK, where K=T or G. This allows for encoding of all potential amino acids (changing their relative representation slightly), but importantly preventing the encoding of two stop residues TAA and TGA.

In one embodiment, the mismatches are fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, etc., or to purines, etc.

As will be appreciated by those in the art, the introduction of a pool of variant targeting polynucleotides (in combination with recombinase) to a target sequence, in vitro to an extrachromosomal sequence, can result in a large number of homologous recombination reactions occuring over time. That is, any number of homologous recombination reactions can occur on a single target sequence, to generate a wide variety of single and multiple mismatches within a single target sequence, and a library of such variant target sequences, most of which will contain mismatches and be different from other members of the library. This thus works to generate a library of mismatches.

In a preferred embodiment, the variant targeting polynucleotides are made to a particular region or domain of a sequence (i.e. a nucleotide sequence that encodes a particular protein domain). For example, it may be desirable to generate a library of all possible variants of a binding domain of a protein, without affecting a different biologically functional domain, etc. Thus, the methods of the present invention find particular use in generating a large number of different variants within a particular region of a sequence, similar to cassette mutagenesis but not limited by sequence length. This idea is sometimes referred to herein as “domain specific gene evolution”. In addition, two or more regions may also be altered simultaneously using these techniques; thus “single domain” and “multi-domain” shuffling can be done. Suitable domains include, but are not limited to, kinase domains, nucleotide-binding sites, DNA binding sites, signaling domains, receptor binding domains, transcriptional activating regions, promoters, origins, leader sequences, terminators, localization signal domains, and, in immunoglobulin genes, the complementarity determining regions (CDR), F_{C}, V_{H} and V_{L}.

In a preferred embodiment, the variant targeting polynucleotides are made to the entire target sequence. In this way, a large number of single and multiple mismatches may be made in an entire sequence.

Thus, this embodiment proceeds as follows, as is generally shown in FIGS. 1 and 4. A pool of targeting polynucleotides are made (termed “combinatorial domain specific gene evolution probes” in the Figures), each containing one or more mismatches. The probes are coated with recombinase as generally described herein, and introduced to the target sequence. Upon binding of the probes to form D-loops, the recombinase is preferably removed. These polynucleotide:target sequences can then introduced into recombinant proficient cells, to produce target protein which can then be tested for biological activity, based on the identification of the target sequence. Depending on the results, the altered target sequence can be used as the starting target sequence in reiterative rounds of homologous recombination, generally using the same library. Preferred embodiments utilize at least two rounds of homologous recombination, with at least 5 rounds being preferred and at least 10 rounds being particularly preferred. Again, the number of reiterative rounds that are performed will depend on the desired end-point, the resistance or succeptibility of the protein to mutation, the number of mismatches in each probe, etc.

In a preferred embodiment, the target sequence is an immunoglobulin. The amino terminal region of the light and heavy chains of an antibody that come together to form the antigen binding site and the variability of their amino acid sequences provides the structural basis for the diversity of antigen binding sites. The variability of the variable regions of both the heavy and light chains is for the most part restricted to three small hypervariable regions in each chain. The remianing part of the variable regions, known as framework regions, is relatively constant. Each of the hypervariable regions consists of only about 5 to 10 amino acids; the corresponding regions in the DNA encoding these regions are known as the complementarity determining regions, or CDRs. Thus to engineer an antibody library, for example an antibody phage library, one can change the sequences in the CDR regions of both the heavy and light chains. Different permutations and combinations of CDRs can be changed and evolved to engineer antibody-phage libraries.

In a preferred embodiment, the target sequence is a single-chain Fv framework for any number of specific antigens. Single chain Fv (scFv) consists of V_{L} and V_{H} domains of an immunoglobulin linked by a peptide spacer and thus contains the minimal antigen-binding domains of an antibody.

In a preferred embodiment, antibody-phage fusions are used as the target sequence. As is known in the art, single-chain Fv fusions with the pill minor coat protein allows expression of the antibody on the surface of a phage, wherein it is available to bind antigen. Five copies of pill are expressed on the surface of the phage. It is therefor possible to express five scFv on the phage. This antibody-phage display system has been used previously to isolate novel antibodies. By starting with antibodies to any antigen, higher affinity antibodies may be made, as well as novel antibodies.

In a preferred embodiment, the target sequence is the coding sequence for β-lactamase.

Thus, the methods of the invention may be used to create superior recombinant reporter genes such as lacZ and green fluorescent protein (GFP); superior antibiotic and drug resistance genes; superior recombinase genes; superior recombinant vectors; and other superior recombinant genes and proteins, including immunoglobulins, vaccines or other proteins with therapeutic value. For example, targeting polynucleotides containing any number of alterations may be made to one or more functional or structural domains of a protein, and then the products of homologous recombination evaluated.

Once made and administered to target cells, the target cells may be screened to identify a cell that contains the targeted sequence modification. This will be done in any number of ways, and will depend on the target gene and targeting polynucleotides as will be appreciated by those in the art. The screen may be based on phenotypic, biochemical, genotypic, or other functional changes, depending on the target sequence. In an additional embodiment, as will be appreciated by those in the art, selectable markers or marker sequences may be included in the targeting polynucleotides to facilitate later identification.

In a preferred embodiment, kits containing the compositions of the invention are provided. The kits include the compositions, particularly those of libraries or pools of degenerate cssDNA probes, along with any number of reagents or buffers, including recombinases, buffers, ATP, etc.

In an alternate embodiment, the targeting polynucleotide:target nucleic acid complexes serve as substrates for single-stranded endonucleases, such as, S1 and mung bean nuclease. Preferably the targeting polynucleotides are substantially complementary and form double D-loops with the target nucleic acid. The junctions of the complexes are single-stranded in nature, and thus are suceptible to single-strand specific nucleases and junction-specific nucleases. Accordingly, treatment of the complex with a single-strand nuclease results in defined nicks in the selected region encoding a predetermined domain of a protein encoded by the target nucleic acid. The nicked target nucleic acid is disassociated from the targeting polynucleotides and are reassembled and “shuffled” in vitro by PCR (Stemmer. 1994. Nature 370:389-391) to produce a plurality modified nucleic acids. The modified nucleic acids are introduced into an appropriate host cell, as described above, for expression of the plurality of modified proteins. Selection techniques are used as described herein to identify and isolate a cell expressing a modified protein. The process is repeated iteratively as needed to further evolve the targeted nucleic acid.

In a preferred embodiment, the present invention finds use in the isolation of new members of gene families that comprise particular domains. As is generally depicted in FIG. 1, the use of domain filaments (i.e. domain homology clamps preferably containing a purification tag such as biotin, disoxisenin, or one purification method such as the use of a RecA antibody), allows the identification of genes containing the domain. Once identified, the new genes can be cloned, sequenced and the protein gene products purified. As will be appreciated by those in the art, the functional importance of the new genes can be assessed in a number of ways, including functional studies on the protein level, as well as the generation of “knock out” animal models. By choosing domain sequences for therapeutically relevant protein domains, novel targets can be identified that can be used in screening of drug candidates.

Thus, in a preferred embodiment, the present invention provides methods for isolating new members of gene families containing protein domains comprising introducing targeting polynucleotides comprising domain homology clamps and at least one purification tag, preferably biotin, to a mix of nucleic acid, such as a plasmid cDNA library or a cell, and then utilizing the purification tag to isolate the gene(s). The exact methods will depend on the purification tag; a preferred method utilizes the attachment of the binding ligand for the tag to a bead, which is then used to pull out the sequence. Alternatively anti-RecA antibodies could be used to capture RecA-coated probes. The genes are then cloned, sequenced, and reassembled if necessary, as is well known in the art.

In an alternate preferred embodiment, the present invention finds use in functional genomic studies, by providing the creation of transgenic animal models of disease. Thus, for example, domain sequences used in homologous recombination methods can generate animals that have a wide variety of mutations in a wide variety of related domains of genes, potentially resulting in a wide variety of phenotypes, including phenotypes related to disease states. That is, by targeting a domain family, one, two or multiple genes in the family may be altered in any given experiment, thus creating a wide variety of genotypes and phenotypes to evaluate. Thus, in a preferred embodiment, the compositions and methods of the invention are used to generate pools or libraries of variant nucleic acid sequences, wherein the mutations are within the functional domain coding region, cellular libraries containing the variant libraries, and libraries of animals containing the variant libraries.

Furthermore, domain targeting can be used in cells or animals that are diseased or altered; in essence, domain targeting can be done to identify “reversion” genes, genes that can modulate disease states caused by domains of different genes. Thus for example the loss of one type of enzymatic activity, resulting in a disease phenotype, may be compensated by alterations in a different but homologous enzymatic activity.

Accordingly, once the recombinase-targeting polynucleotide compositions are formulated, they are introduced or administered into target cells. The administration is typically done as is known for the administration of nucleic acids into cells, and, as those skilled in the art will appreciate, the methods may depend on the choice of the target cell. Suitable methods include, but are not limited to, microinjection, electroporation, lipofection, etc. By “target cells” herein is meant prokaryotic or eukaryotic cells. Suitable prokaryotic cells include, but are not limited to, bacteria such as E. coli, Bacillus species, and the extremophile bacteria such as thermophiles, halophiles, etc. Preferably, the procaryotic target cells are recombination competent. Suitable eukaryotic cells include, but are not limited to, fungi such as yeast and filamentous fungi, including species of Aspergillus, Trichoderma, and Neurospora; plant cells including those of corn, sorghum, tobacco, canola, soybean, cotton, tomato, potato, alfalfa, sunflower, etc.; and animal cells, including fish, reptiles, amphibia, birds and mammals. Suitable fish cells include, but are not limited to, those from species of salmon, trout, tilapia, tuna, carp, flounder, halibut, swordfish, cod and zebrafish. Suitable bird cells include, but are not limited to, those of chickens, ducks, quail, pheasants, ostrich, and turkeys, and other jungle foul or game birds. Suitable mammalian cells include, but are not limited to, cells from horses, cows, buffalo, deer, sheep, rabbits, rodents such as mice, rats, hamsters and guinea pigs, goats, pigs, primates, marine mammals including dolphins and whales, as well as cell lines, such as human cell lines of any tissue or stem cell type, and stem cells, including pluripotent and non-pluripotent, and non-human zygotes. Particular human cells including, but are not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells, osteoclasts, chondrocytes and other connective tissue cells, keratinocytes, melanocytes, liver cells, kidney cells, and adipocytes. Suitable cells also include known research cells, including, but not limited to, Jurkat T cells, mouse La, HT1080, C127, Rat2, CV-1, NIH3T3 cells, CHO, COS, 293 cells, etc. See the ATCC cell line catalog, hereby expressly incorporated by reference.

In a preferred embodiment, procaryotic cells are used to identify, clone, or alter target sequences, preferably protein domains. In this embodiment, a pre-selected target DNA sequence is chosen for alteration. Preferably, the pre-selected target DNA sequence is contained within an extrachromosomal sequence. By “extrachromosomal sequence” herein is meant a sequence separate from the chromosomal or genomic sequences. Preferred extrachromosomal sequences include plasmids (particularly procaryotic plasmids such as bacterial plasmids), p1 vectors, viral genomes, yeast, bacterial and mammalian artificial chromosomes (YAC, BAC and MAC, respectively), and other autonomously self-replicating sequences, although this is not required. As described herein, a recombinase and at least two single stranded targeting polynucleotides which are substantially complementary to each other, each of which contain a homology clamp to the target sequence contained on the extrachromosomal sequence, are added to the extrachromosomal sequence, preferably in vitro. The two single stranded targeting polynucleotides are preferably coated with recombinase, and at least one of the targeting polynucleotides contain at least one nucleotide substitution, insertion or deletion. The targeting polynucleotides then bind to the target sequence in the extrachromosomal sequence to effect homologous recombination and form an altered extrachromosomal sequence which contains the substitution, insertion or deletion. The altered extrachromosomal sequence is then introduced into the procaryotic cell using techniques known in the art. Preferably, the recombinase is removed prior to introduction into the target cell, using techniques known in the art. For example, the reaction may be treated with proteases such as proteinase K, detergents such as SDS, and phenol extraction (including phenol:chloroform:isoamyl alcohol extraction). These methods may also be used for eukaryotic cells. The cells are then grown under conditions which allow the expression of the variant nucleic acids to form variant proteins, particularly with alterations in domains.

In a preferred embodiment, proteins having the desired phenotype are selected and isolated, the modified nucleic acid is sequenced to identify sequences effecting the desired activity, and the process is repeated iteratively as needed to produce a protein having a desired activity or property. Thus, in a preferred embodiment, the methods of the invention are repeated until the desired protein or phenotype is seen.

Alternatively, the pre-selected target DNA sequence is a chromosomal sequence. In this embodiment, the recombinase with the targeting polynucleotides are introduced into the target cell, preferably eukaryotic target cells. In this embodiment, it may be desirable to bind (generally non-covalently) a nuclear localization signal to the targeting polynucleotides to facilitate localization of the complexes in the nucleus. See for example Kido et al., Exper. Cell Res. 198:107-114 (1992), hereby expressly incorporated by reference. The targeting polynucleotides and the recombinase function to effect homologous recombination, resulting in altered chromosomal or genomic sequences.

In a preferred embodiment, eukaryotic cells are used. Basically, any mammalian cells may be used, with mouse, rat, primate and human cells being particularly preferred. Accordingly, suitable cell types include, but are not limited to, tumor cells of all types, i.e., fibroblasts, epithelial cells (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Suitable cells also include known research cells, including, but not limited to, Jurkat T cells, NIH 3T3 cells, CHO, Cos, etc. See the ATCC cell line catalog, hereby expressly incorporated by reference.

For making transgenic non-human animals (which include homologously targeted non-human animals) embryonal stem cells (ES cells), donor cells for nuclear transfer and fertilized zygotes are preferred. In a preferred embodiment, embryonal stem cells are used. Murine ES cells, such as AB-1 line grown on mitotically inactive SNL76/7 cell feeder layers (McMahon and Bradley, Cell 62: 1073-1085 (1990)) essentially as described (Robertson, E. J. (1987) in Teratocarcinomas and Embryonic Stem Cells: A Practical Approach. E. J. Robertson, ed. (oxford: IRL Press), p. 71-112; Zjilstra et al., Nature 342:435-438 (1989); and Schwartzberg et al., Science 246:799-803 (1989), each of which is incorporated herein by reference) may be used for homologous gene targeting. Other suitable ES lines include, but are not limited to, the E14 line (Hooper et al. (1987) Nature 326: 292-295), the D3 line (Doetschman et al. (1985) J. Embryol. Exp. Morph. 87: 21-45), and the CCE line (Robertson et al. (1986) Nature 323: 445-448). The success of generating a mouse line from ES cells bearing a specific targeted mutation depends on the pluripotence of the ES cells (i.e., their ability, once injected into a host blastocyst, to participate in embryogenesis and contribute to the germ cells of the resulting animal).

The pluripotence of any given ES cell line can vary with time in culture and the care with which it has been handled. The only definitive assay for pluripotence is to determine whether the specific population of ES cells to be used for targeting can give rise to chimeras capable of germlne transmission of the ES genome. For this reason, prior to gene targeting, a portion of the parental population of AB-1 cells is injected into C57B1/6J blastocysts to ascertain whether the cells are capable of generating chimeric mice with extensive ES cell contribution and whether the majority of these chimeras can transmit the ES genome to progeny.

In a preferred embodiment, non-human zygotes are used, for example to make transgenic animals, using techniques known in the art (see U.S. Pat. No. 4,873,191; Brinster et al., PNAS 86:7007 (1989); Susulic et al., J. Biol. Chem. 49:29483 (1995), and Cavard et al., Nucleic Acids Res. 16:2099 (1988), hereby incorporated by reference). Preferred zygotes include, but are not limited to, animal zygotes, including fish, avian, reptilian, amphibian and mammalian zygotes. Suitable fish zygotes include, but are not limited to, those from species of salmon, trout, tuna, carp, flounder, halibut, swordfish, cod, tilapia and zebrafish. Suitable bird zygotes include, but are not limited to, those of chickens, ducks, quail, pheasant, turkeys, and other jungle fowl and game birds. Suitable mammalian zygotes include, but are not limited to, cells from horses, cows, buffalo, deer, sheep, rabbits, rodents such as mice, rats, hamsters and guinea pigs, goats, pigs, primates, and marine mammals including dolphins and whales. See Hogan et al., Manipulating the Mouse Embryo (A Laboratory Manual), 2nd Ed. Cold Spring Harbor Press, 1994, incorporated by reference.

The vectors containing the DNA segments of interest can be transferred into the host cell by well-known methods, depending on the type of cellular host. For example, micro-injection is commonly utilized for target cells, although calcium phosphate treatment, electroporation, lipofection, biolistics or viral-based transfection also may be used. Other methods used to transform mammalian cells include the use of Polybrene, protoplast fusion, and others (see, generally, Sambrook et al. Molecular Cloning: A Laboratory Manual, 2d ed., 1989, Cold Spring Harbor Laboratory Press, Cold Spring Harbor, N.Y., which is incorporated herein by reference). Direct injection of DNA and/or recombinase-coated targeting polynucleotides into target cells, such as skeletal or muscle cells also may be used (Wolff et al. (1990) Science 247: 1465, which is incorporated herein by reference).

In a preferred embodiment, the precursor animals or cells already contain a disease allele. As used herein, the term “disease allele” refers to an allele of a gene which is capable of producing a recognizable disease. A disease allele may be dominant or recessive and may produce disease directly or when present in combination with a specific genetic background or pre-existing pathological condition. A disease allele may be present in the gene pool or may be generated de novo in an individual by somatic mutation. For example and not limitation, disease alleles include: activated oncogenes, a sickle cell anemia allele, a Tay-Sachs allele, a cystic fibrosis allele, a Lesch-Nyhan allele, a retinoblastoma-susceptibility allele, a Fabry's disease allele, a Huntington's chorea allele, and a xenoderma pigmentosa allele. As used herein, a disease allele encompasses both alleles associated with human diseases and alleles associated with recognized veterinary diseases. For example, the ΔF508 CFTR allele in a human disease allele which is associated with cystic fibrosis in North Americans.

Once made and administered to target cells, new domains of genes may be isolated as outlined herein.

Alternatively, the target cells may be screened to identify a cell that contains the targeted functional domain sequence modification. This will be done in any number of ways, and will depend on the target domain and targeting polynucleotides as will be appreciated by those in the art. The screen may be based on phenotypic, biochemical, genotypic, or other functional changes, depending on the target sequence. For example, IgE levels may be evaluated for inflammation or asthma; vascular tone or blood pressure can be evaluated for hypertension, behavior screens can be done for neurologic effects, lipoprotein profiles can be screened for cardiovascular effects; secreted molecules can be evaluated for endocrine processes; CBCs can be done for hematology studies, etc. In an additional embodiment, as will be appreciated by those in the art, selectable markers or marker sequences may be included in the targeting polynucleotides to facilitate later identification.

In a preferred embodiment, kits containing the compositions of the invention are provided. The kits include the compositions, particularly those of libraries or pools of degenerate cssDNA probes, along with any number of reagents or buffers, including recombinases, buffers, salts, ATP, etc.

The broad scope of this invention is best understood with reference to the following examples, which are not intended to limit the invention in any manner. All patents, patent applications, and publications cited herein are expressly incorporated by reference in their entirety.

### EXAMPLES

### Example 1

### Domain Specific Gene Evolution of beta-Lactamase

Nucleoprotein filament probes used in this study consist of a library of small DNA oligonucleotides 20-200 base pairs coated with RecA protein. The library of oligonucleotides contain a region of random sequences flanked by homology clamps that hybridize to the domain of beta-lactamase that catalyzes the cleavage of the beta-lactam ring of ampicillin. The library of oligonucleotides are synthesized by standard solid-phase methodologies and purified by electrophoresis on 6% denaturing polyacrylamide gels. The probes are end-labelled by standard methods 32P and/or biotin. Nucleoprotein filaments are formed by the addition of RecA protein to single-stranded DNA (at a ratio of 1 RecA molecule for every 3 nucleotides) as described in Sena and Zarling. 1993. Nature Genet. 3:365-372. Formation of filaments is monitored by a band shift assay in 0.8% agarose gels. In this assay, nucleoprotein filaments formed in the presence of ATPγS give rise to a compact and slower moving band in agarose gels compared to the uncoated ssDNA.

The formation of recombinant hybrids is monitored by standard methods (Golub et al. 1992. Nucl. Acids. Res. 20:3121-3125; Golub et al. 1993. PNAS USA 90:7186-7191; Belotserkovskii et al, 1998; Hsieh et al. 1992. PNAS USA 89:6492-6496; Sena & Zarling, 1993). To form probe-target hybrids the nucleofilaments are mixed with the target DNA, followed by incubation at 37° C. for 1 hour. The mixture is deproteinated by the addition of SDS and the hybrids (probe-target complexes) are separated from free probe and plasmid by electrophoresis on agarose gels. The products are visualized by chemiluminescnence or autoradiography. Resistance to restriction enzyme digestion and transcription is indicative of hybrid formation (Hsieh et al. 1992) and is used to assess hybrid formation both qualitatively and quantitatively.

The hybrid complexes are extracted from the gel and are introduced into ampicillin sensitive, RecA-positive E. coli strains (e.g. BB4) by electroporation. Recombinants with increased levels of beta-lactamase activity are selected by first plating on media with ampicillin at 50 mg/ml to 500 mg/ml and exhibit increased activities of beta-lactamase. The plasmids of individual colonies showing increased resistance are purified and subjected to further rounds of mutagenesis and evolution. Plasmids at each round of selection are isolated from mutant (evolved) cells and sequenced by standard methods to characterize the locations and number of mutations.

### Example 2

### Domain Specific Gene Evolution of Antibody of Botulinum Neurotoxin

Single-chain Fv (scFv) consists of V_{L} and V_{H} domains of an immunoglobulin linked by a peptide spacer and thus contain the minimal antigen-binding domains of an antibody. Fusion proteins between scFv and the pill minor coat protein of phage allows expression of the scFv on the surface of the phage, where it is available to bind antigen. Five copies of pill are expressed on the surface of the phage and, therefore, up to five scFv can be expressed per phage particle. (Barbas et al. PNAS USA 89:4457-4461; Zebedee et al. 1992. PNAS USA 89:3175-3179; Burton et al. 1991. PNAS USA 88:10134-10137)

To produce novel antibodies from phage antibody systems, mice are immunized with heat-inactivated Botulinum toxin. Spleen cell from these animals are used as a source of immunoglobulin mRNA which is reverse transcribed to immunoglobulin cDNA. The antibody V_{L} and V_{H} domains of the cDNA are isolated by PCR and splide together with an appropriate oligonucleotide linker into scFv constructs to produce a library of scFv fusion proteins linked in frame to pill (Pharmacia, Piscataway, N.J.). The scFv phage library is screened to select phage that bind to inactivated Botulinum neurotoxin.

To evolve the scFV to higher affinities, probes are synthesized to target CDRs in the light and heavy chains. Each probe has sequences that are dengerate for corresponding CDRs, but homologous to the frame-work regions for homology clamping (FIG. 8). The probes are combined with RecA to form nucleoprotein filament as described in Example 1. The filament are hybridized to purified scFv phagemid DNA to produce a hybrid complex. Complexes are transformed into recombination proficient E. coli strain (e.g. BB4) to allow strand exchange. The bacteria are also transformed with helper phage to assemble and package the scFv phagement containing mutagenized or evolved CDR regions.

After each round of evolution, scFv-bearing phage are screened for increased affinity binding to Botulinum neurotoxin. Phage bearing high affinity scFV are selected by successively increasing the ionic strength and/or temperature of the binding conditions and measuring dissociation constant. The procedure is performed iteratively to evolve the antibody-phage library. After repeated cyles of mutagenesis, the phage are selected that bind most strongly to the botulinum toxin.

### Example 3

### Domain Specific Gene Evolution of Antibody to Bacillus Spores

Vaccination against antigenic components of Bacillus anthracis, the etiological agent of anthrax, is poorly effective and long term immunity is not conferred. Virulent forms of B. anthracis contain capsular antigenic types of the alpha-polypeptide-D-glutamic acid (most natural amino acids are in the L-form). The presence of this D-glutamic acid polypeptide in capsules eliminates the effective functions of the immune response by inhibiting macrophage phagocytosis of B. anthracis. Gram-positive bacteria (e.g. B. anthracis) also contain teichoic acid as a cell wall component. For development of compositions for preventing and treatingo anthrax, antibody-phage libraries are screened for reactivity against the alpha-polypeptide of the D-glutamic acid and also against the teichoic acid. These phage bearing scFv clones are sequenced to define the CDR and framework regions. Probes are synthesized based on the derived sequences to target the CDR regions of the light and heavy chains of scFv to techoic acid or D-glutamic acid. CDR regions are targeted hybrids are transformed into recombination proficient E. coli as described above with helper phage to allow packaging and expression. After screening to eliminate the non-evolved scFv-phage, the process is reiterated to evolve the library. The evolved scFV are used for the construction of bi-specific antibodies using methods described previously (Mayforth, 1993). One arm of the antigen binding site of this bi-specific antibody will be specific for teichoic acid and the other arm for alpha-polypeptide-D-glutamic acid.

We claim:

1. A method of domain specific gene evolution of a target nucleic acid sequence encoding an amino acid sequence of interest, said method comprising providing a plurality of pairs of single-stranded targeting polynucleotides which are substantially complementary to each other and each comprising a homology clamp that substantially corresponds to or is substantially complementary to a predetermined sequence of said target nucleic acid sequence encoding a domain of said protein, said plurality of pairs comprising a library of mismatches between said targeting polynucleotides and said sequence and a recombinase, to form a library of altered target nucleic acid sequences.

2. A method according to claim 1, further comprising simultaneously or successively providing a second plurality of pairs of single-stranded targeting polynucleotides, which are substantially complementary to each other and are not substantially complementary to said first plurality of polynucleotides and each comprising a second homology clamp, that substantially corresponds to or is substantially complementary to a second predetermined sequence of said target nucleic acid sequence encoding a second domain of said protein, said second plurality of pairs comprising a library of mismatches between said targeting polynucleotides and said second sequence and a recombinase, to form a library of altered target nucleic acid sequences.

3. A method of domain specific gene evolution comprising:
a) combining a target nucleic acid encoding an amino acid sequence of interest with a pair of single-stranded targeting polynucleotides which are substantially complementary to each other and each comprising a homology clamp that substantially corresponds to or is substantially complementary to a predetermined sequence of said nucleic acid encoding a domain of said protein, and a recombinase, to form a recombination intermediate;
b) contacting said intermediate with a single-strand exonuclease or junction-specific nuclease to form a nicked or open ended target nucleic acid; and
c) reassembling and recombining said nicked or open ended target nucleic acid to produce a library of altered target nucleic acids.

4. A method according to claim 3 further comprising:
d) simultaneously or successively combining said target nucleic acid encoding said amino acid sequence of interest with a second pair of single-stranded targeting polynucleotides which are substantially complementary to each other and are not substantially complementary to said first pair of polynucleotides and each comprising a homology clamp that substantially corresponds to or is substantially complementary to a second predetermined sequence of said nucleic acid encoding a second domain of said protein, and a recombinase, to form a recombination intermediate;
e) contacting said intermediate with a single-strand exonuclease or junction-specific nuclease to form a nicked or open ended target nucleic acid; and
f) reassembling and recombining said nicked or open ended target nucleic acid to produce a library of altered target nucleic acids.

5. A method of generating a pool of variant nucleic acid sequences of a pre-selected target nucleic acid sequence in an extrachromosomal sequence, said method comprising:
a) adding to said extrachromosomal sequence at least one recombinase and a plurality of pairs of single-stranded targeting polynucleotides which are substantially complementary to each other and each comprising a homology clamp that substantially corresponds to or is substantially complementary to a preselected target nucleic acid sequence, said plurality of pairs comprising a library of mismatches between said targeting polynucleotide and said target nucleic acid sequence, to form a library of altered extrachromosomal sequences; and
b) repeating step a) on said library of altered extrachromosomal sequences.

6. A method according to claim 5 further comprising:
d) adding simultaneously or successively to said extrachromosomal sequence at least one recombinase and a second plurality of pairs of single-stranded targeting polynucleotides which are substantially complementary to each other and are not substantially complementary to said first plurality of polynucleotides and each comprising a homology clamp that substantially corresponds to or is substantially complementary to a second preselected target nucleic acid sequence, said second plurality of pairs comprising a library of mismatches between said targeting polynucleotide and said second target nucleic acid sequence, to form a library of altered extrachromosomal sequences; and
e) repeating step d) on said library of altered extrachromosomal sequences.

7. A method of generating a pool of variant nucleic acid sequences of a pre-selected target nucleic acid sequence in an chromosomal sequence, said method comprising:
a) adding to said chromosomal sequence at least one recombinase and a plurality of pairs of single-stranded targeting polynucleotides which are substantially complementary to each other and each comprising a homology clamp that substantially corresponds to or is substantially complementary to a preselected target nucleic acid sequence, said plurality of pairs comprising a library of mismatches between said targeting polynucleotide and said target nucleic acid sequence, to form a library of altered chromosomal sequences; and
b) repeating step a) on said library of altered extrachromosomal sequences.

8. A method according to claim 7 further comprising:
d) adding simultaneously or successively to said chromosomal sequence at least one recombinase and a second plurality of pairs of single-stranded targeting polynucleotides which are substantially complementary to each other and are not substantially complementary to said first plurality of polynucleotides and each comprising a homology clamp that substantially corresponds to or is substantially complementary to a second preselected target nucleic acid sequence, said second plurality of pairs comprising a library of mismatches between said targeting polynucleotide and said second target nucleic acid sequence, to form a library of altered chromosomal sequences; and
e) repeating step d) on said library of altered chromosomal sequences.

9. A method according to claim 1, 2, 3, or 4 further comprising repeating said method on said library of altered target nucleic acid sequences.

10. A method according to claim 1, 2, 3, 4, 5, 6, 7, or 8 further comprising introducing said library of altered target nucleic acid sequences into cells to form a cellular library comprising variant nucleic acid sequences.

11. A method according to claim 10 further comprising expressing said library of altered target nucleic acid sequences to generate a pool of variant amino acid sequences.

12. A method according to claim 10 or 11 further comprising selecting a cell comprising an altered target nucleic acid sequence having a desired activity.

13. A method according to claim 10 or 11 further comprising selecting a cell comprising an altered target nucleic acid sequence having a desired phenotype.

14. A method according to claim 11 further comprising secreting said pool of variant amino acid sequences.

15. A method according to claim 10, 11, 12, or 13 wherein said recombinase is removed prior to said introducing.

16. A method according to claim 1, 2, 3, 4, 5, 6, 7, 8, 9, 10, 11, 12, 13, 14, or 15 wherein said cells are eukaryotic.

17. A method according to claim 1, 2, 3, 4, 5, 6, 7, 8, 9, 10, 11, 12, 13, 14, 15, or 16 wherein said cells are procaryotic.

18. A method according to claim 1, 2, 3, 4, 5, 6, 7, 8, 9, 10, 11, 12, 13, 14, 15, 16, or 17 wherein said targeting polynucleotides are coated with said recombinase.

19. A method according to claim 1, 2, 3, 4, 5, 6, 7, 8, 9, 10, 11, 12, 13, 14, 15, 16, 17, or 18 wherein said recombinase is a species of prokaryotic recombinase.

20. A method according to claim 1, 2, 3, 4, 5, 6, 7, 8, 9, 10, 11, 12, 13, 14, 15, 16, 17, or 18 wherein said recombinase is a species of eukaryotic recombinase.

21. A method according to claim 11, wherein the variant amino acid sequences comprise a plurality of amino acid substitutions.

22. A method according to claim 1, 2, 3, 4, 5, 6, 7, 8, 9, 10, 11, 12, 13, 14, 15, 16, 17, 18, 19, 20, or 21 wherein at least one of said complementary single stranded nucleic acids further comprise a chemical substituent.

23. A method according to claim 1, 2, 3, 4, 5, 6, 7, 8, 9, 10, 11, 12, 13, 14, 15, 16, 17, 18, 19, 20, 21, or 22 wherein the target amino acid sequence comprises a complementary determining region.

24. A method according to claim claim 1, 2, 3, 4, 5, 6, 7, 8, 9, 10, 11, 12, 13, 14, 15, 16, 17, 18, 19, 20, 21, 22, or 23 wherein said target nucleic acid sequence comprises an expression vector.


SEQUENCE LISTING (6 sequences)

SEQ ID NO: 1 - DNA, 80 bp, Artificial Sequence
  Synthetic
tcctgcaaggcttctggctactccttcaccnnnnnnnnnnnnnnctgggtgaagcagaggcctggacaaggccttgagtg

SEQ ID NO: 2 - DNA, 113 bp, Artificial Sequence
  Synthetic
aggcctggacaaggccttgagtggattggcnnnnnnnnnnnnnnnnnnnnnnnnnnnnnnnnnnnnnnnnnnnnnnnnnnnaaggccacattgactgtagacagatcctccag

SEQ ID NO: 3 - DNA, 102 bp, Artificial Sequence
  Synthetic
gactctgcggtctattactgtgctagagggnnnnnnnnnnnnnnnnnnnnnnnnnnnnnnnnnnnnnnnnnnntggggccaagggaccacggcaccgtctcc

SEQ ID NO: 4 - DNA, 88 bp, Artificial Sequence
  Synthetic
tctccaggggagaaggtcatcatgacctgcnnnnnnnnnnnnnnnnnnnnnnnnnnntactggtaccagcagaagccaggatcctccc

SEQ ID NO: 5 - DNA, 84 bp, Artificial Sequence
  Synthetic
ccaggatcctcccccagactcctgatttatnnnnnnnnnnnnnnnnnnnnnnnngtccctgttcgcttcagtggcagtgggtct

SEQ ID NO: 6 - DNA, 91 bp, Artificial Sequence
  Synthetic
ggaggctgaagatgctgccacttattactgcnnnnnnnnnnnnnnnnnnnnnnnnnnnnnnggtgctgggacaaagctggagctcaaacgt